(19) United States
(12) Patent Application Publication
(10) Pub. No.: US 2018/0161069 A1
(43) Pub. Date: Jun. 14, 2018

(54) SUBLAMINAR SPINAL FIXATION SYSTEM AND METHOD OF USE

(71) Applicant: Christian DiPaola, Shrewsbury, MA (US)

(72) Inventor: Christian DiPaola, Shrewsbury, MA (US)

(21) Appl. No.: 15/786,796

(22) Filed: Oct. 18, 2017

Related U.S. Application Data
(60) Provisional application No. 62/409,671, filed on Oct. 18, 2016.
(60) Provisional application No. 62/569,112, filed on Oct. 6, 2017.

(51) Int. Cl. A61B 17/70

(52) U.S. Cl. CPC A61B 17/7032; A61B 17/7019; A61B 17/707; A61B 17/7053

(57) ABSTRACT

A spinal fixation system that utilizes the sublaminar approach is disclosed. Devices and methods describe herein generate a link with two secure attachments (one to the spine and one to the spinal fixation rod) that can be manipulated separately from one another.

### REFERENCE TO PENDING PRIOR PATENT APPLICATIONS

This patent application:

(i) claims benefit of pending prior U.S. Provisional Patent Application Ser. No. 62/409,671, filed Oct. 18, 2016 by Christian DiPaola for SUBLAMINAR SPINAL FIXATION SYSTEM AND METHOD OF USE (Attorney's Docket No. DIPAOLA-1 PROV); and

(ii) claims benefit of pending prior U.S. Provisional Patent Application Ser. No. 62/569,112, filed Oct. 6, 2017 by Christian DiPaola for SUBLAMINAR SPINAL FIXATION SYSTEM AND METHOD OF USE (Attorney's Docket No. DIPAOLA-3 PROV).

The two (2) above-identified patent applications are hereby incorporated herein by reference.

### FIELD OF THE INVENTION

This invention relates to medical implants in general, and more particularly to spinal implants.

### BACKGROUND OF THE INVENTION

Looking first at FIG. 1, the human spine comprises a plurality of vertebrae that are normally aligned along a vertical axis. As seen in FIG. 2, each vertebra generally comprises a vertebral body, a pair of pedicles, a pair of transverse processes, a lamina (sometimes also referred to as the posterior arch), a pair of superior facets, a pair of inferior facets and a spinous process.

In some cases, an individual's spine may not be properly aligned along a vertical axis. By way of example but not limitation, and looking now at FIG. 3, in a scoliotic spine, the spine may present an abnormal curvature. In this case, it may be necessary to straighten the spine so as to eliminate the abnormal curvature from the spine. This is typically done by securing spinal fixation rods to the spine so as to force the vertebrae into the desired configuration.

More particularly, pedicle spinal fixation systems generally comprise spinal fixation rods that are anchored to the pedicles of the vertebrae using mounts and screws.

Additionally, sublaminar spinal fixation systems generally comprise spinal fixation rods that are anchored to the laminae of the vertebrae using mounts and sublaminar bands (or cables or wires, etc.). More particularly, the sublaminar bands are wrapped around the lamina, and the mounts connect the spinal fixation rods to the sublaminar bands.

Unfortunately, current sublaminar spinal fixation systems do not allow the mounts to be connected and tensioned to the lamina, and the spinal fixation rods to be reduced to the mounts, in separate and distinct operations. Rather, with current sublaminar spinal fixation systems, the mounts are tensioned to the lamina, and the spinal fixation rods are reduced to the mounts, in a single, simultaneous tensioning step. This does not allow for optimal tensioning of the bands, or for optimal positioning of the mounts, or for optimal reduction and positioning of the spinal fixation rods. In addition, in some cases, when the sublaminar bands and spinal fixation rods are simultaneously tensioned, excessive stress can be applied to the bone-implant interface. This can result in significant trauma to the patient, complicate the surgery, and cause device failure.

Current sublaminar spinal fixation systems also do not allow the mounts to be fixed to the spine in their final tensioned states independently of the spinal fixation rod and hence independently of one another.

And current sublaminar spinal fixation systems do not allow the spinal fixation rod to be reduced to the spine (mount), or the spine reduced to the rod (mount), without an instrument engaging the sublaminar band. Put another way, current systems do not allow fixation of the mount to the spine without instruments engaging the band. The band cannot be tensioned and cut prior to the rod reduction step.

Thus there is a need for a new and improved sublaminar spinal fixation system which allows the sublaminar bands to be tensioned and locked to the mounts, and the spinal fixation rods to be reduced and locked to the mounts (or the mounts to be reduced and locked to the spinal fixation rods), in separate and distinct operations.

### SUMMARY OF THE INVENTION

The present invention comprises the provision and use of a new and improved sublaminar spinal fixation system which allows the sublaminar bands to be tensioned and locked to the mounts, and the spinal fixation rods to be reduced and locked to the mounts (or the mounts to be reduced and locked to the spinal fixation rods), in separate and distinct operations.

As will hereinafter be discussed, one or more aspects of the present invention allows independent tensioning of the sublaminar bands and locking to the mount, and independent reduction of the spinal fixation rods and locking to the mount. This allows for more precise control of the spine, and deformities can be corrected in a more controlled fashion than with current sublaminar spinal fixation systems.

And one or more aspects of the present invention generates two secure attachments (one from the spine to the mount via the sublaminar bands, and one from the spinal fixation rod to the mount) which can be manipulated separately from one another, yet are integrated into one unified sublaminar spinal fixation system.

And one or more aspects of the present invention generates a separate connection between the sublaminar band and the mount, and a separate connection between the spinal fixation rod and the mount.

And one or more aspects of the present invention locks the sublaminar band to the mount independently of the locking mechanism that attaches the spinal fixation rod to the mount.

And one or more aspects of the present invention allows the surgeon to tension and lock all of the mounts to the spine in a complete and finished step prior to proceeding with the reduction of the spinal fixation rod to the mounts (or the reduction of the mounts to the spinal fixation rod).

And one or more aspects of the present invention allows the spinal fixation rod to be reduced to the mounts, or vice versa, without the surgeon and/or his/her instruments engaging the sublaminar bands. The present system allows reduction and/or fixation of the rod to the mount without instruments engaging the band. Also, the band can be tensioned and cut prior to the rod reduction step.

In one preferred form of the invention, there is provided a method for spinal repair, the method comprising:

position a band having a first end and a second end around at least a portion of a bone;

positioning a mount having independent first and second coupling members, the first coupling member for coupling the mount to the band and the second coupling member for coupling a spinal fixation rod to the mount;

coupling the band to the mount using the first coupling member;

tensioning at least one of the ends of the band;

while maintaining tension on the band, fixing the mount to the band using the first coupling member, wherein the first coupling member fixes the mount to the band by advancing toward the at least a portion of the bone; and

reducing the distance between the spinal fixation rod and the mount;

wherein the step of reducing the distance between the spinal fixation rod and the mount is achieved without tensioning the band; and

wherein the step of reducing the distance between the spinal fixation rod and the mount does not change the location or orientation of the mount relative to the bone.

In another preferred form of the present invention, there is provided a spinal fixation system, the spinal fixation system comprising:

a band;

a spinal fixation rod; and

a mount, wherein the mount comprises a first connection mechanism for connecting the band to the mount, and a second connection mechanism for connecting the spinal fixation rod to the mount, wherein the first and second connection mechanisms operate independently of one another, and further wherein the first connection mechanism connects the band to the mount by advancing a member away from the second connection mechanism, and further wherein the first connection mechanism and the second connection mechanism are disposed in vertical alignment with one another.

In another preferred form of the present invention, there is provided a spinal fixation system, the spinal fixation system comprising:

a mount;

a band having first and second ends, wherein the first end of the band is fixed to the mount and the second end of the band comprises a free end selectively fixable to the mount; and

a spinal fixation rod;

wherein the mount comprises a first connection mechanism for connecting the second end of the band to the mount; and

wherein the mount comprises a second connection mechanism for connecting the spinal fixation rod to the mount.

In another preferred form of the present invention, there is provided a method for spinal repair, the method comprising:

providing a spinal fixation system, the spinal fixation system comprising:
a mount;
a band having first and second ends, wherein the first end of the band is fixed to the mount and the second end of the band comprises a free end selectively fixable to the mount; and
a spinal fixation rod;
wherein the mount comprises a first connection mechanism for connecting the second end of the band to the mount; and
wherein the mount comprises a second connection mechanism for connecting the spinal fixation rod to the mount;

passing the second end of the band around at least a portion of a bone;

connecting the second end of the band to the mount using the first connection mechanism so as to secure the mount to the bone; and

connecting the spinal fixation rod to the mount using the second connection mechanism.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a normal spine;

FIG. 2 is a schematic view showing a vertebra;

FIG. 3 is a schematic view showing a scoliotic spine;

FIG. 4 is a schematic view of a novel sublaminar spinal fixation system formed in accordance with the present invention;

FIGS. 5-8 are schematic views showing the mount of the sublaminar spinal fixation system shown in FIG. 4;

FIGS. 9-12 are schematic views showing a band tensioning tool which may be used with the sublaminar spinal fixation system of the present invention;

FIG. 13 is a schematic view of a rod reduction tool which may be used with the sublaminar spinal fixation system of the present invention;

FIGS. 14-17 are schematic views showing a sublaminar band and mount being secured to the lamina of a vertebra in accordance with the present invention;

FIGS. 18-20 are schematic views showing a spinal fixation rod being secured to a mount (which has been previously secured to a sublaminar band) in accordance with the present invention;

FIG. 21 is a schematic representation of selected aspects of the present invention;

FIG. 22 is a schematic view of still another mount formed in accordance with the present invention;

FIGS. 23-25 are schematic views showing details of a portion of the mount shown in FIG. 22;

FIG. 26 is a schematic view of still another mount formed in accordance with the present invention;

FIGS. 27-32 are schematic views showing another novel sublaminar spinal fixation system formed in accordance with the present invention;

FIGS. 33-38 are schematic views showing another novel sublaminar spinal fixation system formed in accordance with the present invention;

FIGS. 39-42 are schematic views showing another novel sublaminar spinal fixation system formed in accordance with the present invention; and

FIGS. 43-49 are schematic views showing another novel sublaminar spinal fixation system formed in accordance with the present invention.

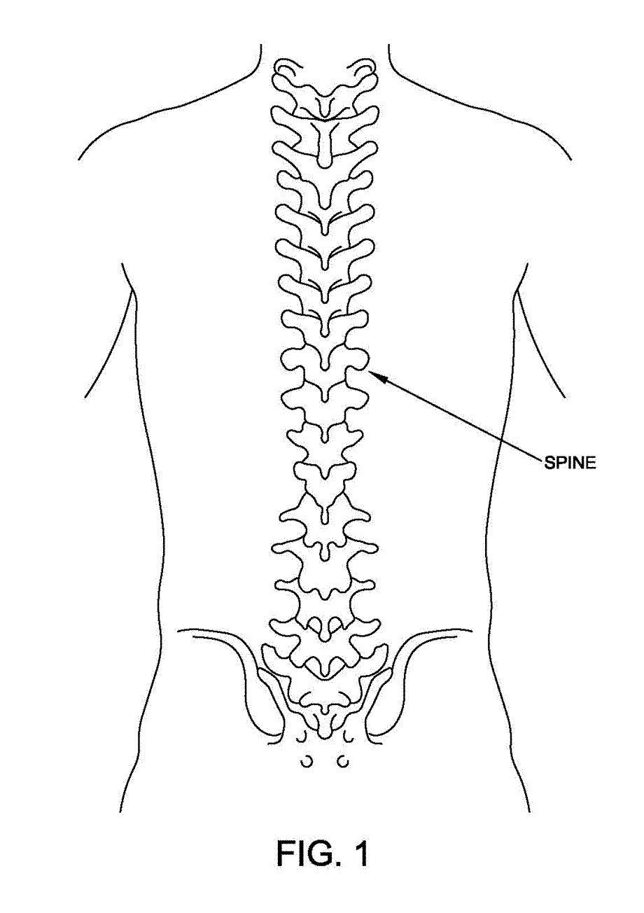

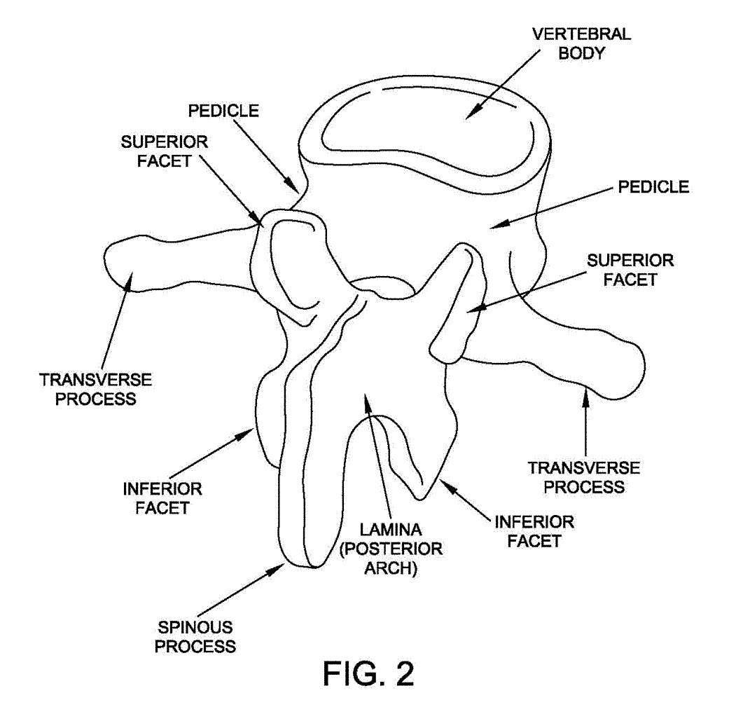

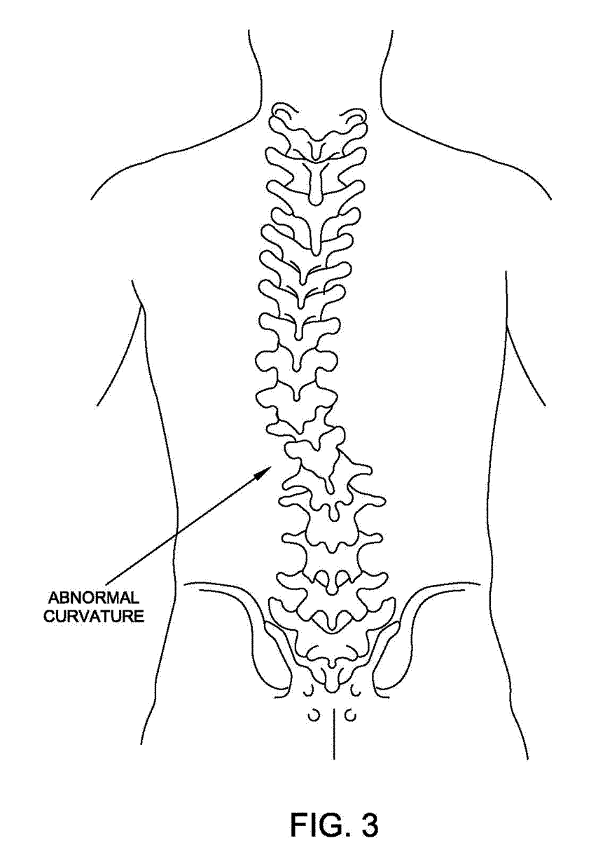

### DETAILED DESCRIPTION OF THE PREFERRED EMBODIMENTS

The present invention comprises the provision and use of a new and improved sublaminar spinal fixation system which allows the sublaminar bands to be tensioned and locked to the mounts, and the spinal fixation rods to be reduced and locked to the mounts (or the mounts to be reduced and locked to the spinal fixation rods), in separate and distinct operations. The present system allows reduction and/or fixation of the rod to the mount without instruments engaging the band. Also, the band can be tensioned and cut prior to the rod reduction step.

As will hereinafter be discussed, one or more aspects of the present invention allows independent tensioning of the sublaminar bands and locking to the mount, and independent reduction of the spinal fixation rods and locking to the mount. This allows more precise control of the spine, and deformities can be corrected in a more controlled fashion than with current sublaminar spinal fixation systems.

And one or more aspects of the present invention generates two secure attachments (one from the spine to the mount via the sublaminar bands, and one from the spinal fixation rod to the mount) which can be manipulated separately from one another, yet are integrated into one unified sublaminar spinal fixation system.

And one or more aspects of the present invention generates a separate connection between the sublaminar band and the mount, and a separate connection between the spinal fixation rod and the mount.

And one or more aspects of the present invention locks the sublaminar band to the mount independently of the locking mechanism that attaches the spinal fixation rod to the mount.

And one or more aspects of the present invention allows the surgeon to tension and lock all of the mounts to the spine in a complete and finished step prior to proceeding with the reduction of the spinal fixation rod to the mounts (or the reduction of the mounts to the spinal fixation rod).

And one or more aspects of the present invention allows the spinal fixation rod to be reduced to the mounts, or vice versa, without the surgeon and/or his/her instruments engaging the sublaminar bands.

### The Novel Sublaminar Spinal Fixation System

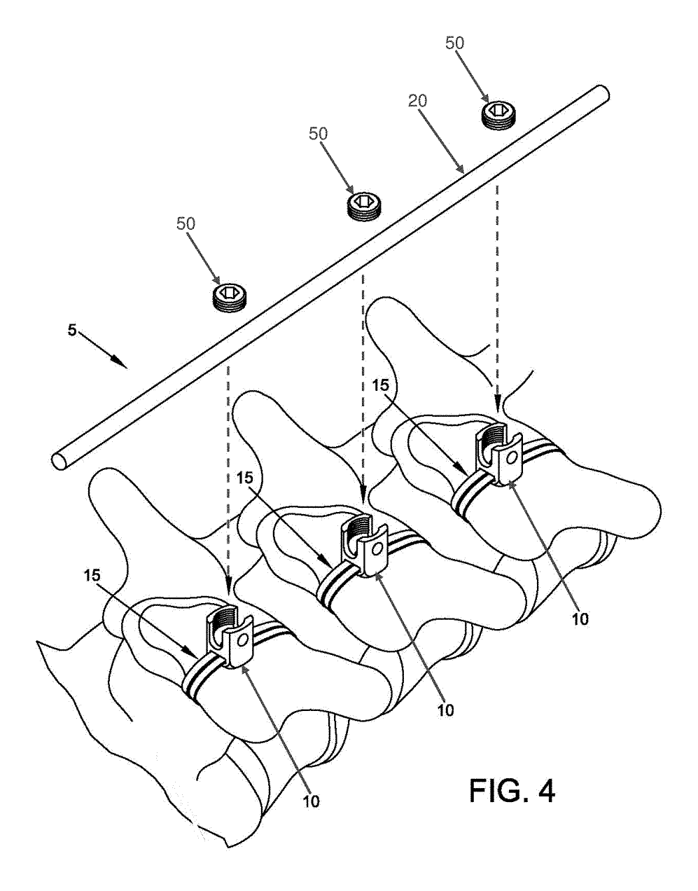

Looking first at FIG. 4, there is shown a novel sublaminar spinal fixation system 5. Sublaminar spinal fixation system 5 generally comprises at least one mount 10, at least one sublaminar band 15, and at least one spinal fixation rod 20. As will hereinafter be discussed, sublaminar bands 15 connect mounts 10 to the laminae of vertebrae, and spinal fixation rods 20 are secured to mounts 10. It should be appreciated that, as will hereinafter be discussed, sublaminar bands 15 may also connect mounts 10 to other parts of the vertebrae (e.g., to the spinous processes of the vertebrae, the transverse processes of the vertebrae, etc.), or to other anatomical structures, including non-spinal structures (e.g., to other bones, to soft tissue, etc.), or to prostheses which may themselves be mounted to anatomical structures (e.g., to prosthetic lamina, to prosthetic transverse processes, to screws, etc.). To this end, while band 15 is frequently referred to herein as a “sublaminar band”, this term is only intended to facilitate an understanding of a primary application of band 15, and the term “sublaminar band” is not intended to convey any limitation to the application of band 15 and no such limitation should be inferred.

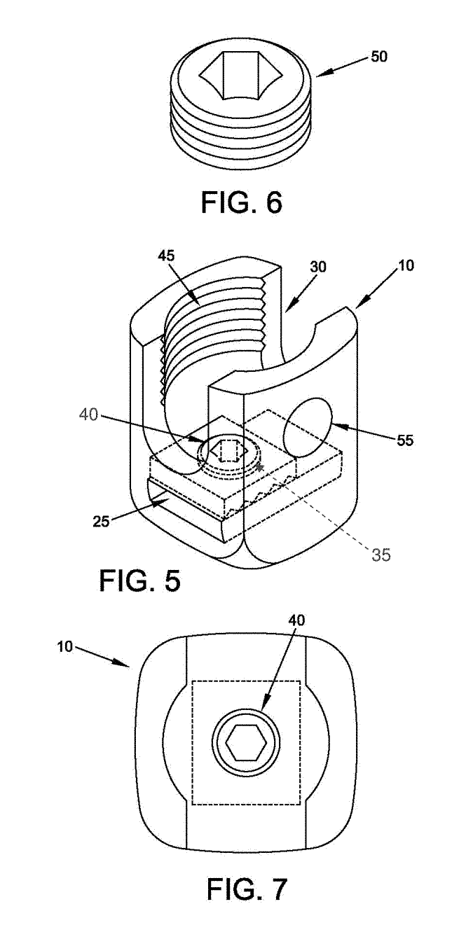

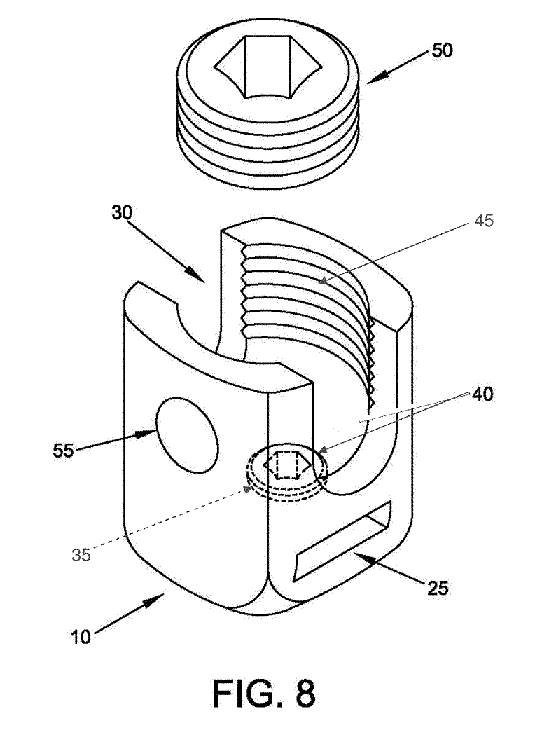

One preferred form of mount 10 is shown in FIGS. 5-8. Mount 10 generally comprises a bottom slot 25 for receiving a sublaminar band 15 (FIG. 4), a U-shaped slot 30 for receiving a spinal fixation rod 20 (FIG. 4), a threaded bore 35 communicating with bottom slot 25 for receiving a headless screw 40 for securing a sublaminar band 15 to mount 10, and a threaded bore 45 communicating with U-shaped slot 30 for receiving a headless screw 50 for securing a spinal fixation rod 20 to mount 10. Mount 10 also comprises one or more connection elements 55 (e.g., a recess, a flange, etc.) for connection to a band tensioning tool (see below) and/or a rod reduction tool (see below). In one preferred form of the invention, connection elements 55 comprise industry-standard connection elements of the sort used to connect a band tensioning tool and/or a rod reduction tool to a conventional mount (or head) of a pedicle-based spinal fixation system. Note also that the mounts may be designed such that the head or the rod-accepting element contains, or is comprised of, extended tabs or walls that are threaded. These extended walls or tabs act as reducing elements that a reduction tool threads down so as to aid in the reduction of the rod. The tabs or walls are removed once the rod is secured to the mount, prior to finishing the surgery.

Sublaminar band 15 comprises a strong, flexible biocompatible material. By way of example but not limitation, sublaminar band 15 may comprise a polyester band, a Kevlar band, a braided titanium cable, etc.

Spinal fixation rod 20 may comprise a conventional spinal fixation rod of the sort well known in the art.

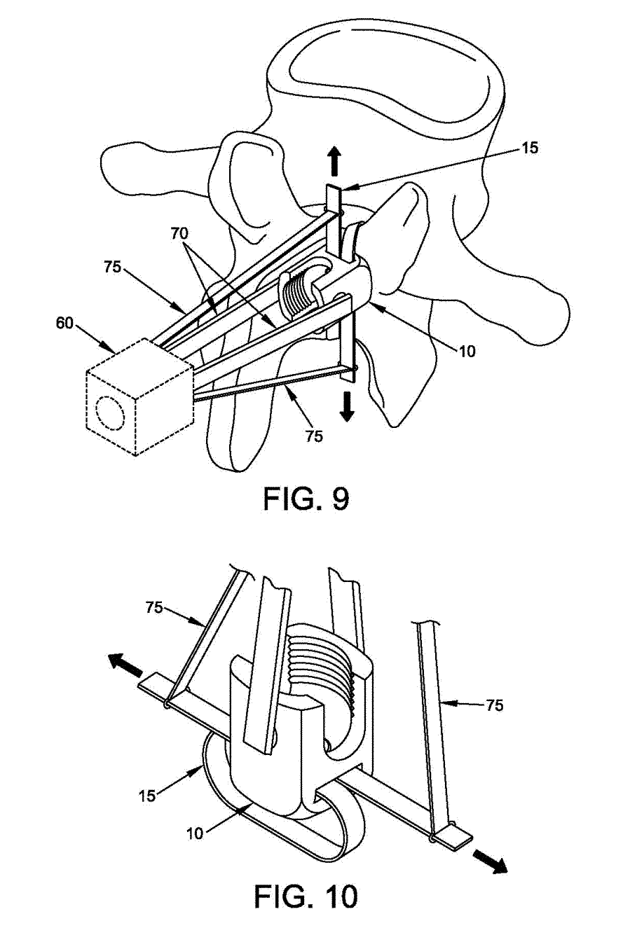

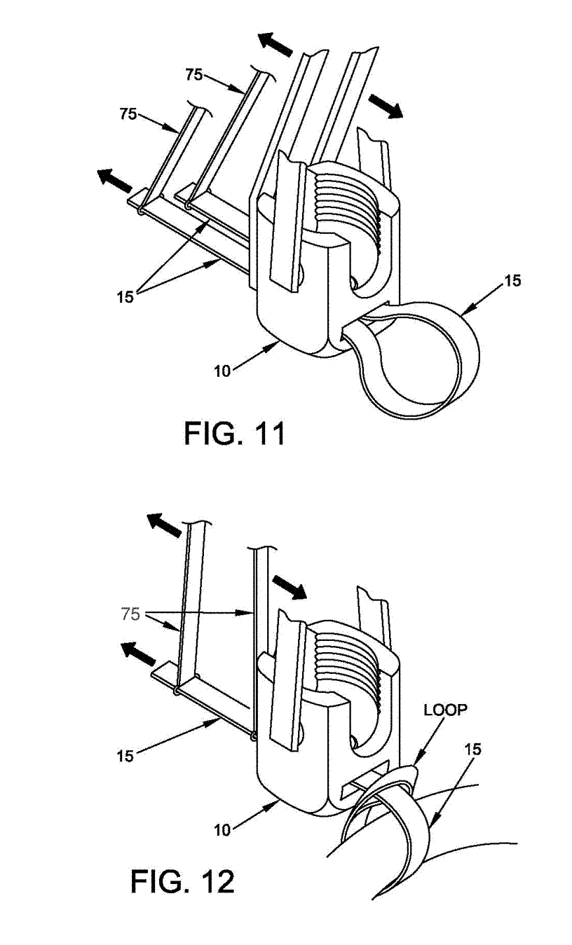

Looking next at FIGS. 9-12, there is shown a band tensioning tool 60 for tensioning sublaminar band 15 after the sublaminar band has been passed around the lamina of a vertebrae (or other anatomical or prosthetic structure) and through bottom slot 25 of mount 10. Band tensioning tool 60 generally comprises one or more legs 70 for connecting to connection elements 55 of mount 10, and two arms 75 for securing to the opposing ends of sublaminar band 15 and applying tension to the sublaminar band.

It will be appreciated that sublaminar band 15 may be passed around the lamina (or other anatomical or prosthetic structure) and through bottom slot 25 of mount 10 with various configurations, and to this end band tensioning tool 60 may have different configurations. By way of example but not limitation, where the two free ends of sublaminar band 15 are passed through bottom slot 25 of mount 10 in opposing directions (FIGS. 9 and 10), arms 75 may be configured to pull in opposing directions; where the two free ends of sublaminar band 15 are passed through bottom slot 25 in a parallel direction (FIG. 11), arms 75 may be configured to pull in a parallel direction; and where one free end of sublaminar band 15 is looped and the other free end of sublaminar band 15 is passed through that loop (FIG. 12), one arm 75 may be configured to pull away from mount 10.

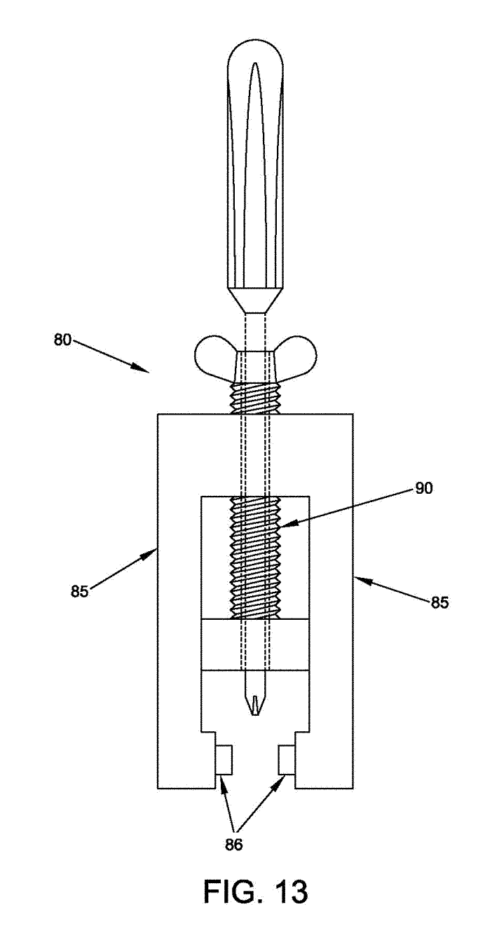

Looking next at FIG. 13, there is shown a rod reduction tool 80 for reducing spinal fixation rod 20 to mount 10 (or for reducing mount 10 to spinal fixation rod 20) after sublaminar band 15 has been tensioned and secured to mount 10. Rod reduction tool 80 generally comprises one or more legs 85 having connection elements 86 for connecting to connection elements 55 of mount 10, and a screw press 90 (or other force-applying device of the sort well known in the art, e.g., a ratchet mechanism) for reducing spinal fixation rod 20 to mount 10 (or vice versa).

### Use of the Novel Sublaminar Spinal Fixation System

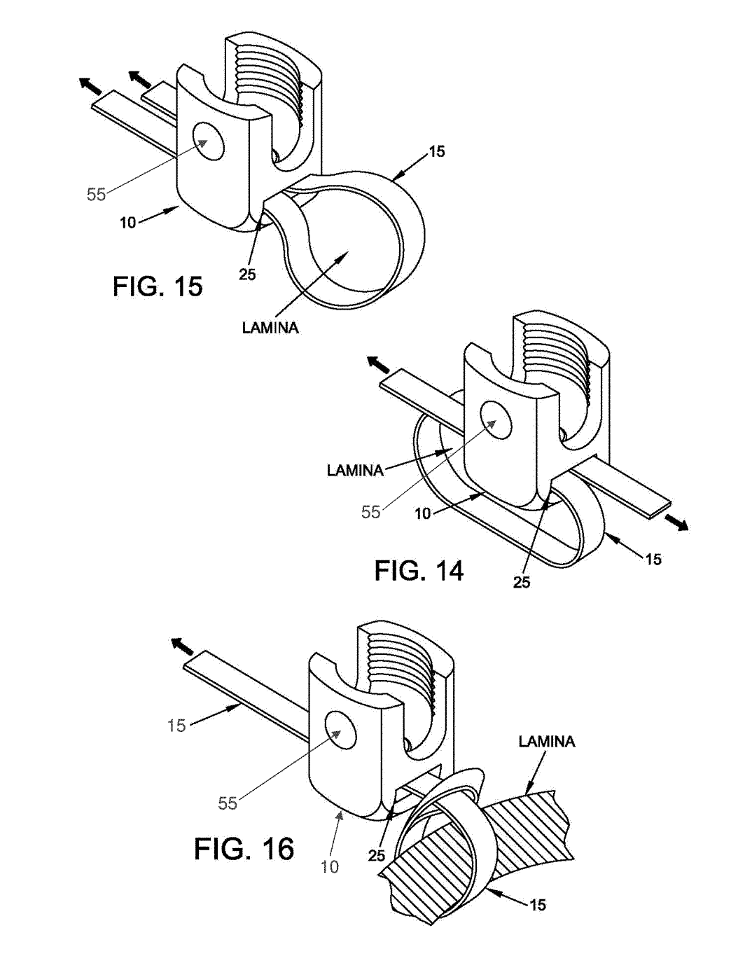

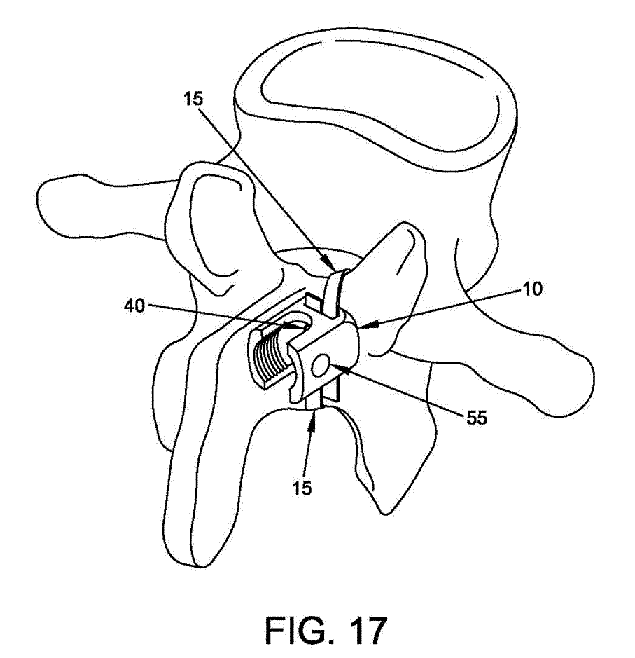

In one intended method of use, a sublaminar band 15 is first passed around the lamina of a vertebra (or other anatomical or prosthetic structure) and through bottom slot 25 of mount 10. As noted above, there are various ways for passing sublaminar band 15 around the lamina of a vertebra and through bottom slot 25 of mount 10. By way of example but not limitation, the two free ends of sublaminar band 15 may be passed through bottom slot 25 of mount 10 in opposing directions (FIG. 14), or the two free ends of sublaminar band 15 may be passed through bottom slot 25 of mount 10 in a parallel direction (FIG. 15), or one free end of sublaminar band 15 may be looped and the other free end of sublaminar band 15 may be passed through that loop (FIG. 16).

Once sublaminar band 15 has been passed around the lamina of a vertebra (or other anatomical or prosthetic structure) and through bottom slot 25 of mount 10, the sublaminar band is tensioned and secured to mount 10. More particularly, depending on how sublaminar band 15 has been passed around the lamina of a vertebra (or other anatomical or prosthetic structure) and through bottom slot 25 of mount 10 (FIGS. 14-16), an appropriate band tensioning tool 60 is used to tension the sublaminar band (see FIGS. 9-12). Then headless screw 40 is inserted into threaded bore 35 so as to secure the tensioned sublaminar band 15 to mount 10. See FIG. 17. Note that sublaminar band 15 is tensioned to the lamina (or other anatomical or prosthetic structure) and mount 10, and then secured to the lamina and mount 10, independently of any reduction of spinal fixation rod 20 to mount 10 (or any reduction of mount 10 to spinal rod 20).

If desired, sublaminar band 15 may then be trimmed to length by removing any excess ends extending from mount 10.

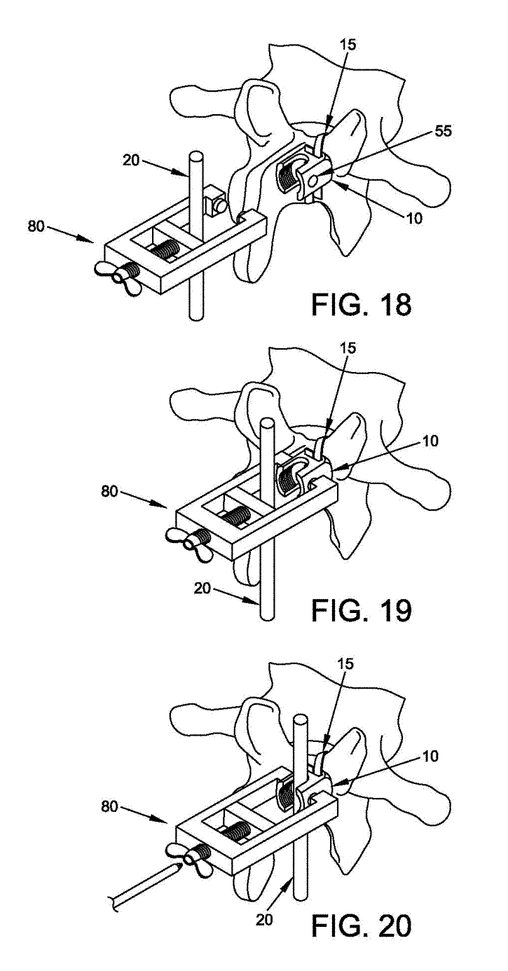

Next, spinal fixation rod 20 is reduced and secured to mount 10. This may be done by straddling spinal fixation rod 20 with legs 85 of rod reduction tool 80 (FIG. 18), securing legs 85 of rod reduction tool 80 to connection elements 55 of mount 10 (FIG. 19), and then using screw press 90 (or other force-applying device of the sort well known in the art, e.g., a ratchet mechanism) of rod tensioning tool 80 to reduce spinal fixation rod 20 into U-shaped slot 30 of mount 10 (or to reduce mount 10 to spinal fixation rod 20). Alternatively, rod reduction tool 80 may be secured to mount 10, spinal fixation rod 20 inserted between legs 85 of rod reduction tool 80, and then screw press 90 (or other force-applying device of the sort well known in the art, e.g., a ratchet mechanism) of rod reduction tool 80 used to reduce spinal fixation rod 20 into U-shaped slot 30 of mount 10 (FIG. 20) (or to reduce mount 10 to spinal fixation rod 10). In either case, once spinal fixation rod 20 has been reduced into U-shaped slot 30 of mount 10 (or mount 10 reduced to spinal fixation rod 20), headless screw 50 may be advanced into threaded bore 45 of mount 10 so as to secure spinal fixation rod 20 to mount 10. Note that spinal fixation rod 20 is reduced to mount 10 (or mount 10 reduced to spinal fixation rod 20), and then the spinal fixation rod is secured to mount 10, independently of any tensioning and securing of sublaminar band 15 to mount 10. Indeed, sublaminar band 15 does not need to be engaged in any way by the surgeon and/or his/her instruments during the time that the spinal fixation rod 20 is being reduced to mount 10 (or vice versa).

If sublaminar band 15 has not already been trimmed to length, it may be trimmed to length at this point in the procedure.

Significantly, because the sublaminar spinal fixation system of the present invention allows the mounts to be tensioned to the lamina (or other anatomical or prosthetic structure), and the spinal fixation rods to be reduced to the mounts (or vice versa), in separate and independent operations, optimal positioning of the mounts, and optimal reduction and positioning of the spinal fixation rods, is facilitated. In addition, because the sublaminar spinal fixation system of the present invention allows the sublaminar bands and spinal fixation rods to be separately and independently tensioned, excessive stress on the bone-implant interface can be avoided. This can minimize trauma to the patient, simplify the surgery, and reduce device failure.

It should also be appreciated that inasmuch as the sublaminar spinal fixation system of the present invention allows the mounts to be tensioned and fixed to the lamina (or other anatomical or prosthetic structure), and the spinal fixation rods to be reduced to the mounts (or vice versa), in separate and independent operations, the surgeon has greater flexibility in conducting the surgery. By way of example but not limitation, if desired, the surgeon can deploy (i.e., position and tension) a plurality (or all) of the mounts to the laminae of the vertebrae (or other anatomical or prosthetic structures), and then, after some or all of the mounts have been deployed, reduce and secure the spinal fixation rod to the previously-deployed mounts (or reduce the previously-deployed mounts to the spinal fixation rod). This approach is popular with spinal fixation systems which use pedicle screws to secure the mounts to the vertebrae, and is not possible with conventional sublaminar spinal fixation systems (which simultaneously tension the mounts to the lamina, and reduce the spinal fixation rod to the mounts, in a singular operation).

Essentially, the present invention allows the surgeon to attach some or all of the mounts to the spine and he/she may move on to other parts of the operation such as placing pedicle screws or performing osteotomies. Since the mounts can be independently and initially tensioned and locked to the spine, the surgeon can obtain all fixation points to the spine in a finalized stable configuration (for example, pedicle screws, hooks and sublaminar implants) prior to the insertion and/or reduction of the rod to the fixation points. With the present invention, the surgeon can focus on the step of rod reduction to the fixation points (whether they be all sublaminar mounts or a hybrid of sublaminar implants, hooks and/or screws) as a singular step utilizing a sequential application of similar reduction instruments. Current systems do not allow this.

And it should be appreciated that inasmuch as mount 10 has the connection mechanism for spinal fixation rod 20 (i.e., U-shaped slot 30 and threaded bore 45/headless screw 50) disposed “in-line” (i.e., in vertical alignment) with the connection mechanism for sublaminar band 15 (i.e., bottom slot 25 and threaded bore 35/headless screw 40), mount 10 can have a smaller “footprint” which allows the surgeon greater latitude in proper anatomical positioning of the spinal fixation system.

### Schematic Representation

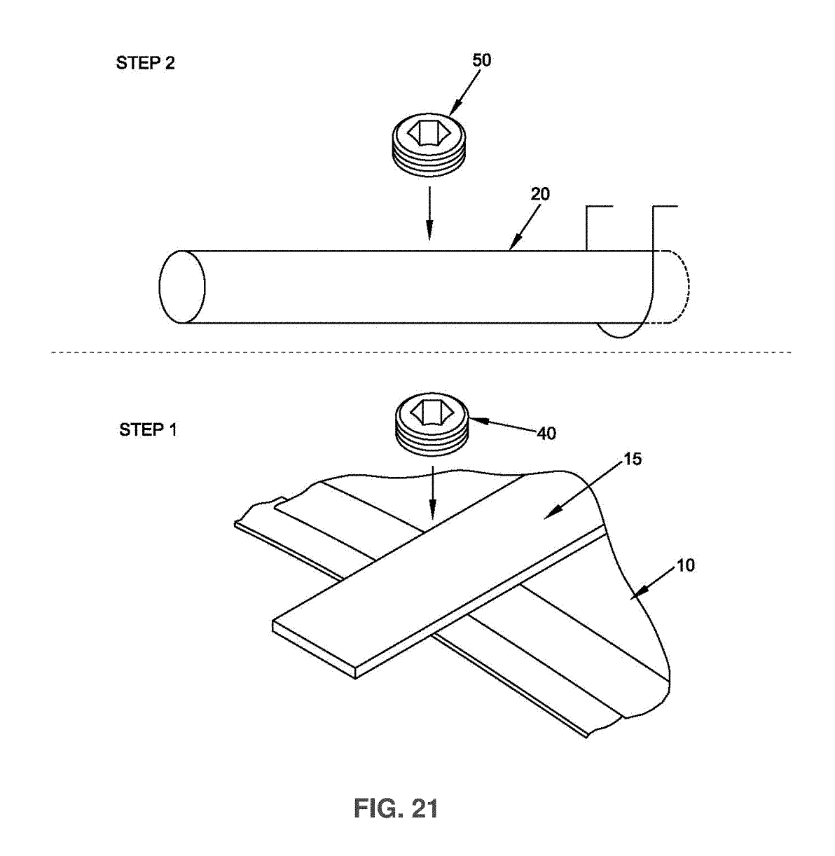

FIG. 21 is a schematic representation of several significant aspects of the present invention. More particularly, with the present invention, sublaminar band 15 is tensioned and secured to mount 10 in a first separate and independent operation, and then spinal fixation rod 20 is reduced and secured to mount 10 (or mount 10 is reduced and secured to spinal fixation rod 20) in a second separate and independent operation. And in one form of the present invention, mount 10 is constructed so that the connection mechanism for spinal fixation rod 20 is disposed “in-line” (i.e., in vertical alignment; also known as coaxial) with the connection mechanism of sublaminar band 15.

### “Lateral Connector” Mounts

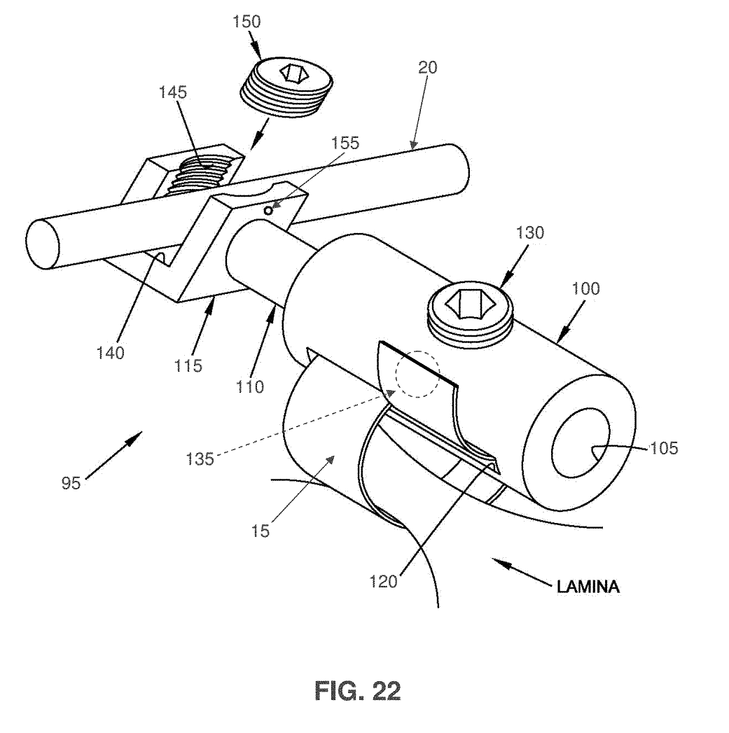

Looking next at FIG. 22, there is shown a “lateral connector” mount 95 also formed in accordance with the present invention. Mount 95 generally comprises a body 100 having an opening 105 formed therein, a connector 110 mounted in opening 105, and a body 115 mounted to connector 110. Connector 110 allows body 115 to be adjustably positioned relative to body 100; at the same time, connector 110 links body 115 with body 100 so that the two bodies behave as a unified structure.

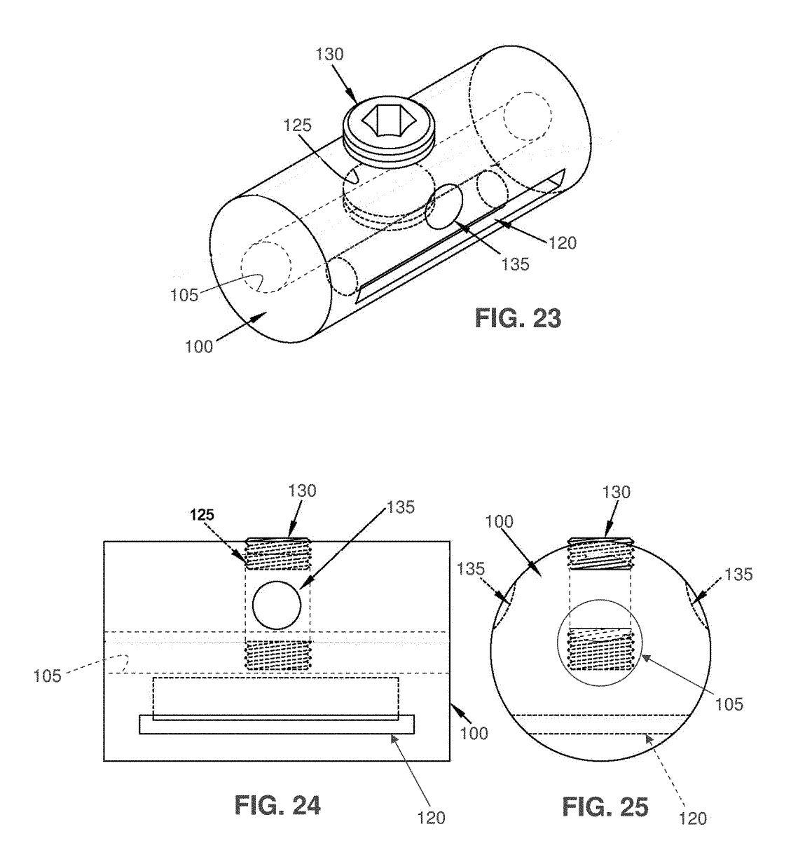

Body 100 is shown in greater detail in FIGS. 23-25. In addition to opening 105, body 100 generally comprises a bottom slot 120 for receiving sublaminar band 15, a threaded bore 125 communicating with bottom slot 120 for receiving a headless screw 130 for securing sublaminar band 15 to body 100. Body 100 also comprises one or more connection elements 135 (e.g., a recess, a flange, etc.) for connection to a band tensioning tool (see above).

Body 115 has a U-shaped slot 140 for receiving a spinal fixation rod 20, and a threaded bore 145 communicating with U-shaped slot 140 for receiving a headless screw 150 for securing spinal fixation rod 20 to body 115. Body 115 also comprises one or more connection elements 155 (e.g., a recess, a flange, etc.) for connection to a rod reduction tool (see above).

In use, sublaminar band 15 is first passed around the lamina of a vertebra (or other anatomical or prosthetic structure) and through bottom slot 120 of body 100. An appropriate band tensioning tool, e.g., band tensioning tool 60 disclosed above, is used (in conjunction with connection elements 135 of body 100) to tension sublaminar band 15. Then headless screw 130 is inserted into threaded bore 125 so as to secure sublaminar band 15 to body 100. Note that sublaminar band 15 is tensioned to the lamina (or other anatomical or prosthetic structure) and body 100, and then secured to the lamina (or other anatomical or prosthetic structure) and body 100, independently of any reduction of spinal fixation rod 20 to body 115.

If desired, sublaminar band 15 may then be trimmed to length by removing any excess ends extending from body 100.

Next, spinal fixation rod 20 is reduced and secured to body 115 (or body 115 is reduced to spinal fixation rod 20). This is done using a rod reduction tool, e.g., rod reduction tool 80 disclosed above, in conjunction with connection elements 155, to reduce spinal fixation rod 20 to body 115, and then inserting spinal fixation rod 20 into U-shaped (or other appropriately accommodative-shaped) slot 140 (or reducing body 115 to spinal fixation rod 20). Then headless screw 150 is advanced into threaded bore 145 to secure spinal fixation rod 20 to body 115. Note that spinal fixation rod 20 is reduced to body 115, and then secured to body 115, or body 115 is reduced to spinal fixation rod 20 and then secured to spinal fixation rod 20, independently of any tensioning and securing of sublaminar band 15 to body 100. Indeed, sublaminar band 15 does not need to be engaged in any way by the surgeon and/or his/her instruments as spinal fixation rod 20 is reduced to body 115 (or vice versa).

If sublaminar band 15 has not already been trimmed to length, it may be trimmed to length at this point in the procedure.

Note that with the construction shown in FIGS. 22-25, mounts 95 are still tensioned to the lamina (or other anatomical or prosthetic structure), and the spinal fixation rod reduced to the mounts (or vice versa), in separate and independent operations, whereby to provide the advantages discussed above with respect to the construction of FIGS. 4-21. Note also that with the construction shown in FIGS. 22-25, the connection mechanism for spinal fixation rod 20 is no longer disposed “in-line” (i.e., in vertical alignment) with the connection mechanism for sublaminar band 15. While this construction does not provide as small a “footprint” as the constructions shown in FIGS. 4-21, it can be highly useful where some lateral offset is desired between the connection mechanism for spinal fixation rod 20 and the connection mechanism for sublaminar band 15.

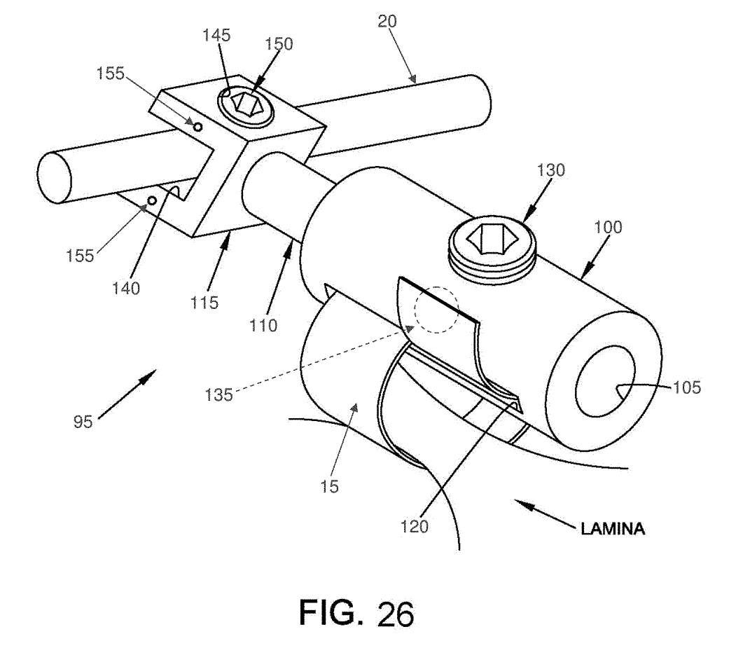

FIG. 26 shows another “lateral connector” mount generally similar to the “lateral connector” mount 95 shown in FIG. 22, except that the U-shaped slot 140 is oriented in a different direction.

### Additional Comments

As noted above, while a primary application of sublaminar bands 15 is to secure mounts 10 to the laminae of the vertebrae, it should be appreciated that sublaminar bands 15 may also connect mounts 10 to other parts of the vertebrae (e.g., to the spinous processes of the vertebrae, the transverse processes of the vertebrae, etc.), or to other anatomical structures, including non-spinal structures (e.g., to other bones, to soft tissue, etc.), or to prostheses which may themselves be mounted to anatomical structures (e.g., to prosthetic lamina, to prosthetic transverse processes, etc.). As also noted above, while band 15 is frequently referred to herein as a “sublaminar band”, this term is only intended to facilitate an understanding of a primary application of band 15, and the term “sublaminar band” is not intended to convey any limitation to the application of band 15 and no such limitation should be inferred.

In addition to the foregoing, while the present invention has sometimes been discussed above in the context of straightening the spine to treat scoliosis, it should also be appreciated that the present invention may be used in substantially any orthopedic application where a fixation rod must be secured to a bony structure (or other anatomical or prosthetic structure) for the purposes of treating a patient. By way of example but not limitation, the present invention may be used for other spine pathologies including, but not limited to, trauma, oncology, infection or degenerative applications in which spinal fixation is required.

### Mounts Having One End of the Sublaminar Band Permanently Fixed to the Mount and the Other End of the Sublaminar Band Selectively Fixable to the Mount

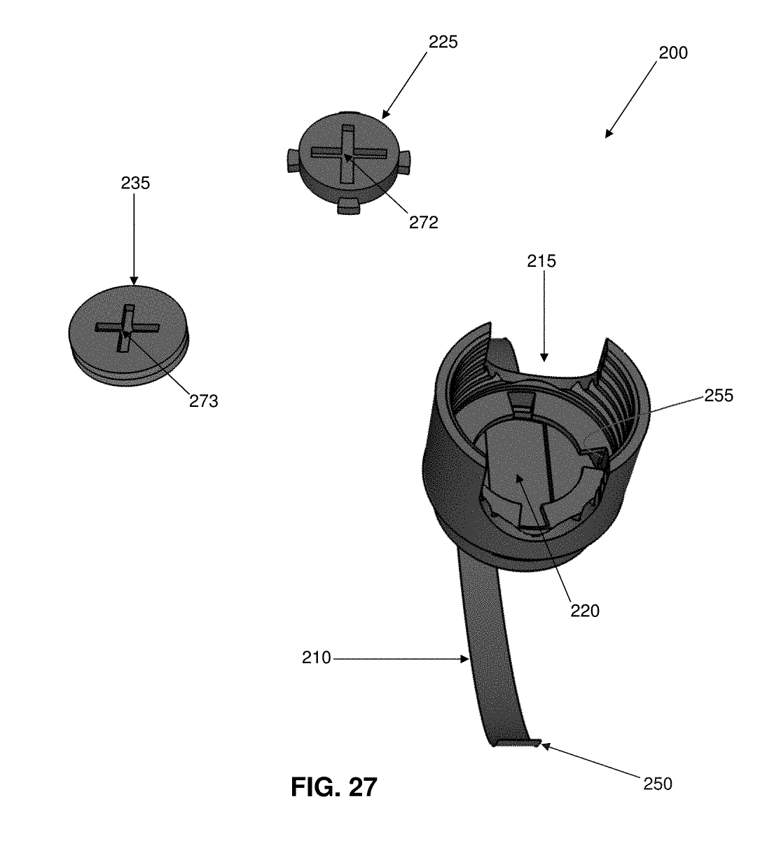

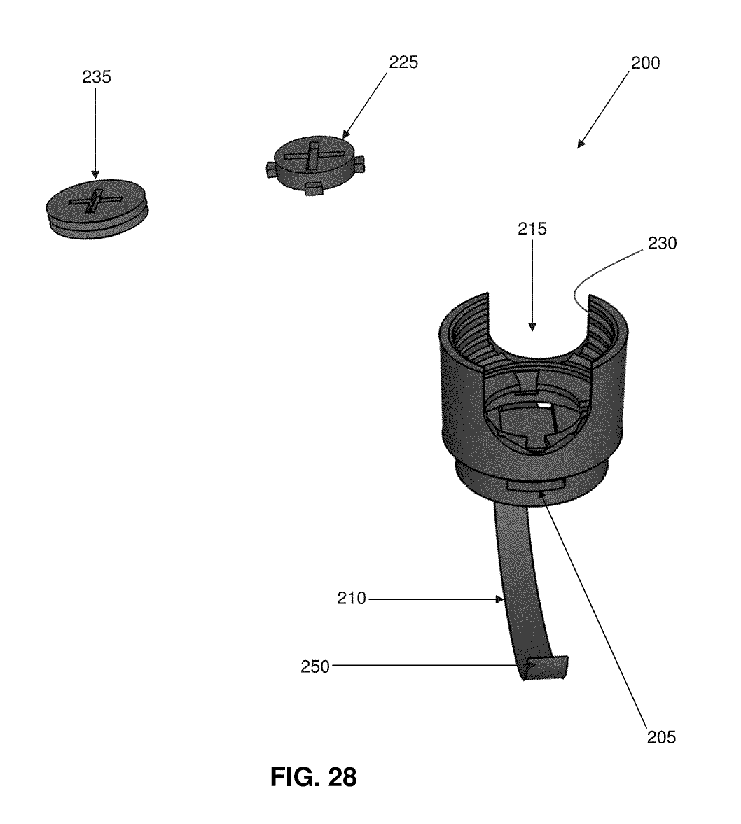

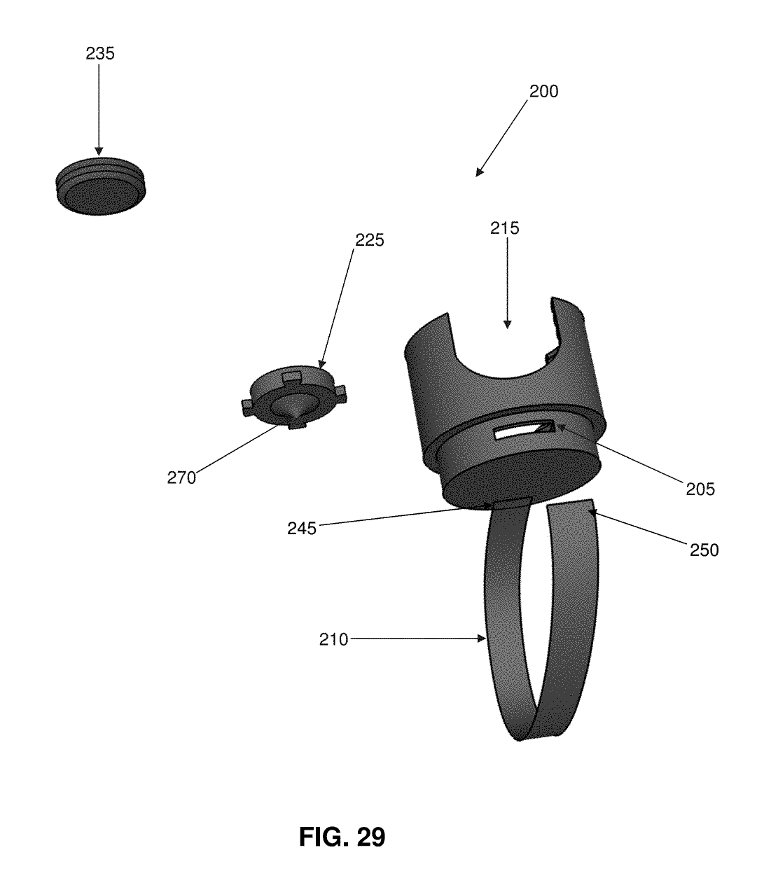

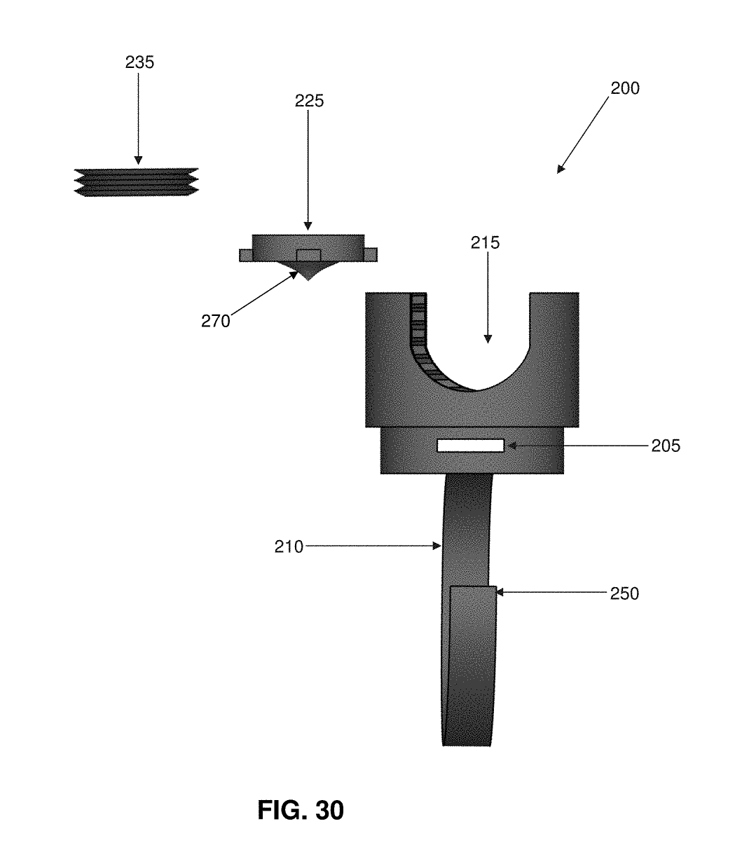

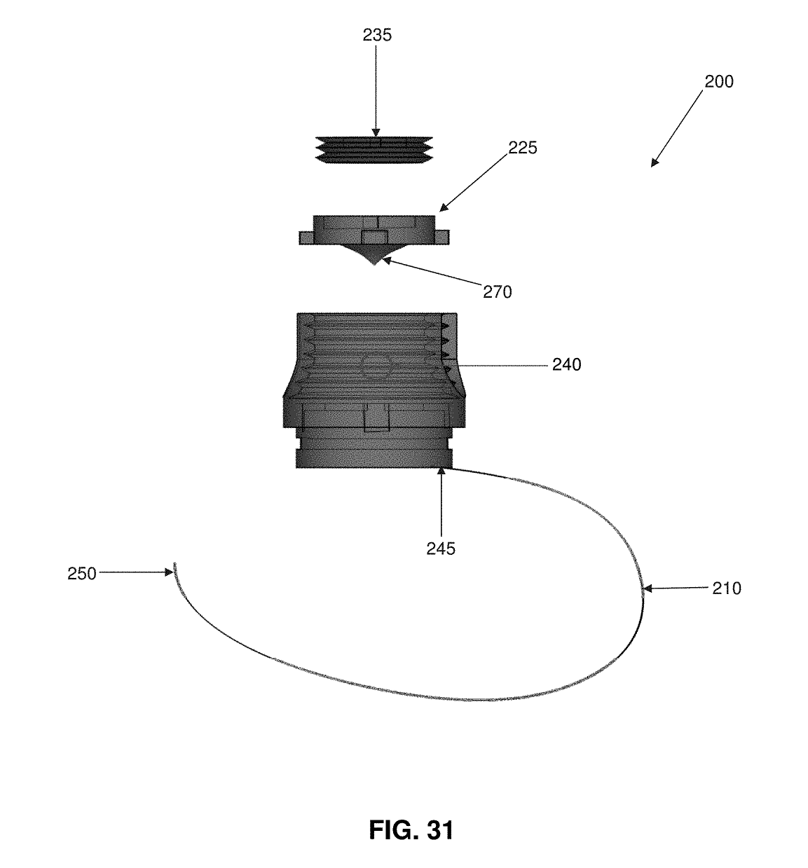

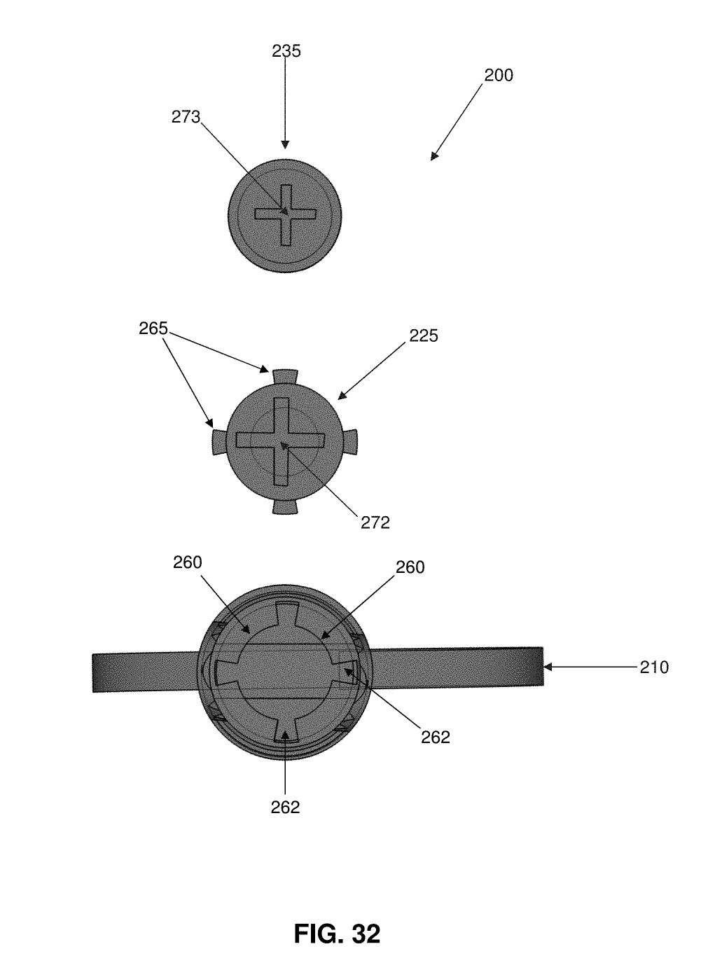

Looking next at FIGS. 27-32, there is shown a construction wherein one end of the sublaminar band is permanently fixed to the mount and the other end of the sublaminar band is selectively fixable to the mount.

More particularly, in this form of the invention, there is provided a mount 200 which generally comprises a bottom slot 205 for receiving the “free end” of a sublaminar band 210, a U-shaped slot 215 for receiving spinal fixation rod 20 (FIG. 4), a bayonet mount 220 disposed adjacent to bottom slot 205 for receiving a headless fastener 225 (for securing sublaminar band 210 to mount 200), and a threaded bore 230 disposed about U-shaped slot 215 for receiving a headless screw 235 (for securing spinal fixation rod 20 to mount 200).

Mount 200 also comprises one or more connection elements 240 (e.g., a recess, a flange, etc.) for connection to a band tensioning tool (see above) and/or a rod reduction tool (see above). In one preferred form of the invention, connection elements 240 comprise industry-standard connection elements of the sort used to connect a band tensioning tool and/or a rod reduction tool to a conventional mount (or head) of a pedicle-based spinal fixation system.

Sublaminar band 210 comprises a fixed end 245 and a “free end” 250. Fixed end 245 of sublaminar band 210 is permanently fixed to mount 200. Free end 250 of sublaminar band 210 is configured to be received by bottom slot 205 of mount 200 and selectively held in bottom slot 205 by headless fastener 225. In one preferred form of the present invention, sublaminar band 210 comprises a strong, flexible biocompatible material. By way of example but not limitation, sublaminar band 210 may comprise a polyester band, a Kevlar band, a braided titanium cable, etc.

In one preferred form of the invention, free end 250 of sublaminar band 210 may be stiffened somewhat so as to facilitate passage of free end 250 (and thus sublaminar band 210) around the lamina and into bottom slot 205 of mount 200. At the same time, free end 250 of sublaminar band 210 remains sufficiently flexible so that it can still be configured into a C-shaped loop. It should be appreciated that free end 250 of sublaminar band 210 may be stiffened in a variety of ways, e.g., by placing a stiffening material inside free end 250, by placing a stiffening material over free end 250, etc.

Bayonet mount 220 is configured to receive headless fastener 225 so that headless fastener 225 can secure free end 250 of sublaminar band 210 to mount 200. More particularly, bayonet mount 220 comprises a recess 255, a plurality of flanges 260, and a plurality of openings 262 disposed between the plurality of flanges 260.

Headless fastener 225 comprises a plurality of tabs 265, a spike 270 and a non-circular recess 272. The plurality of tabs 265 are sized so as to be passable through the plurality of openings 262 of bayonet mount 220 and rotatable into recess 255 of bayonet mount 220 so that the plurality of tabs 265 may be locked below the plurality of flanges 260 of bayonet mount 220. A non-circular recess 272 (e.g., a cruciform recess) may be used to turn headless fastener 225 (see below).

In one form of the invention, spike 270 may be replaced by a blunt element. If desired, the blunt element may comprise a projection, teeth, ridges, an abrasive region, etc. in order to increase the securement of the blunt element to sublaminar band 210 in bottom slot 205.

Headless screw 235 is sized so as to fit into threaded bore 230. A non-circular recess 273 (e.g., a cruciform recess) may be used to turn headless screw 235 (see below).

In use, mount 200 is first positioned adjacent to the lamina of a vertebra (or other anatomical or prosthetic structure) and free end 250 of sublaminar band 210 is passed around the lamina of the vertebra (or other anatomical or prosthetic structure). Then free end 250 of sublaminar band 210 is passed through bottom slot 205 of mount 200.

Once free end 250 of sublaminar band 210 has been passed around the lamina of the vertebra (or other anatomical or prosthetic structure) and free end 250 of sublaminar band 210 has been passed through bottom slot 205 of mount 200, sublaminar band 210 is tensioned and secured to mount 200. More particularly, an appropriate band tensioning tool 60 (see above) is used to tension free end 250 of sublaminar band 210. Then headless fastener 225 is inserted into bayonet mount 220, with the plurality of tabs 265 of headless fastener 225 aligned with the plurality of openings 262 of bayonet mount 220. The plurality of tabs 265 of headless fastener 225 are then inserted through the plurality of openings 262 of bayonet mount 220 so that the plurality of tabs 265 lie in recess 255 of bayonet mount 220. Once the plurality of tabs 265 of headless fastener 225 are in recess 255 of bayonet mount 220, headless fastener 225 is turned (e.g., using non-circular recess 272) so as to position the plurality of tabs 265 of headless fastener 225 beneath the plurality of flanges 260 of bayonet mount 220, thereby locking headless fastener 225 to bayonet mount 220, and thus locking headless fastener 225 to mount 200. When locked in this position, spike 270 of headless fastener 225 presses into sublaminar band 210 so as to secure the tensioned sublaminar band 210 to mount 200. It should be appreciated that where spike 270 is replaced with a blunt element, the blunt element compresses sublaminar band 210 in bottom slot 205 so as to secure the tensioned sublaminar band 210 to mount 200.

Note that sublaminar band 210 is tensioned to the lamina (or other anatomical or prosthetic structure) and mount 200, and then secured to the lamina and mount 200, independently of any reduction of spinal fixation rod 20 to mount 200 (or any reduction of mount 200 to spinal rod 20).

If desired, sublaminar band 210 may then be trimmed to length by removing any excess of free end 250 extending from bottom slot 205 of mount 200.

Next, spinal fixation rod 20 is reduced and secured to mount 200. This is done by reducing spinal fixation rod 20 into U-shaped slot 215 of mount 200 (or to reduce mount 200 to spinal fixation rod 20). Once spinal fixation rod 20 has been reduced into U-shaped slot 215 of mount 200 (or mount 200 reduced to spinal fixation rod 20), headless screw 235 may be rotatably advanced (e.g., using non-circular recess 273) into threaded bore 230 of mount 200 so as to secure spinal fixation rod 20 to mount 200. Note that spinal fixation rod 20 is reduced to mount 200 (or mount 200 reduced to spinal fixation rod 20), and then the spinal fixation rod is secured to mount 200, independently of any tensioning and securing of sublaminar band 210 to mount 200. Indeed, sublaminar band 210 does not need to be engaged in any way by the surgeon and/or his/her instruments during the time that the spinal fixation rod 20 is being reduced to mount 200 (or vice versa).

If sublaminar band 210 has not already been trimmed to length, it may be trimmed to length at this point in the procedure.

It should be appreciated that since the mounts 200 can be independently and initially tensioned and locked to the spine, the surgeon can obtain all fixation points to the spine in a finalized stable configuration (for example, pedicle screws, hooks and sublaminar implants) prior to the insertion and/or reduction of the spinal fixation rod to the fixation points.

It should also be appreciated that inasmuch as mount 200 has the connection mechanism for spinal fixation rod 20 (i.e., U-shaped slot 215 and threaded bore 230/headless screw 235) disposed “in-line” (i.e., in vertical alignment) with the connection mechanism for sublaminar band 210 (i.e., bottom slot 205 and bayonet mount 220/headless fastener 225), mount 200 can have a smaller “footprint” which allows the surgeon greater latitude in proper anatomical positioning of the mounts 200.

It should also be appreciated that, if desired, the connection mechanism for spinal fixation rod 20 (i.e., U-shaped slot 215 and threaded bore 230/headless screw 235) need not be aligned with the connection mechanism for sublaminar band 210 (i.e., bottom slot 205 and bayonet mount 220/headless fastener 225). If desired, U-shaped slot 215 may be laterally offset from the axis of bayonet mount 220 in order to accommodate anatomical needs.

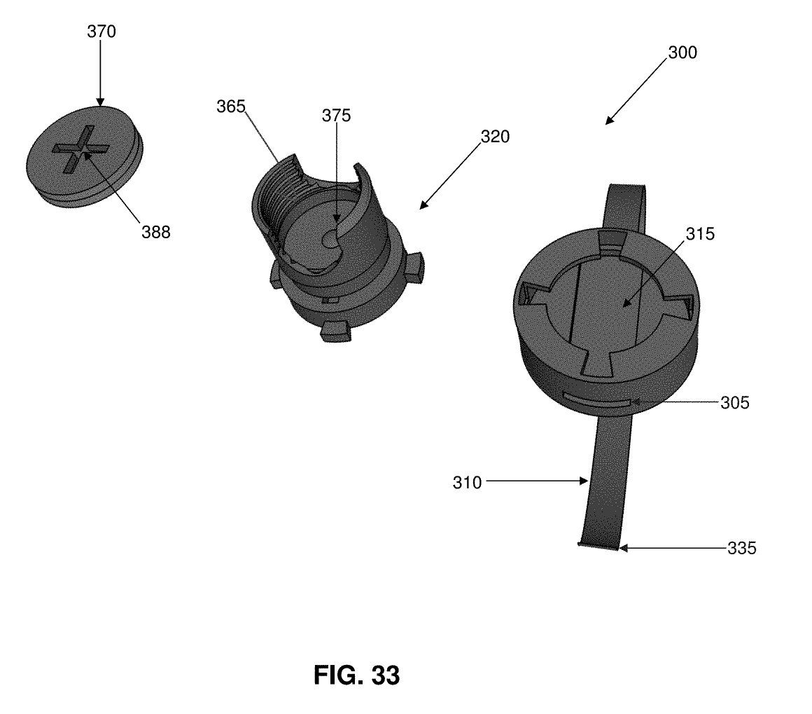

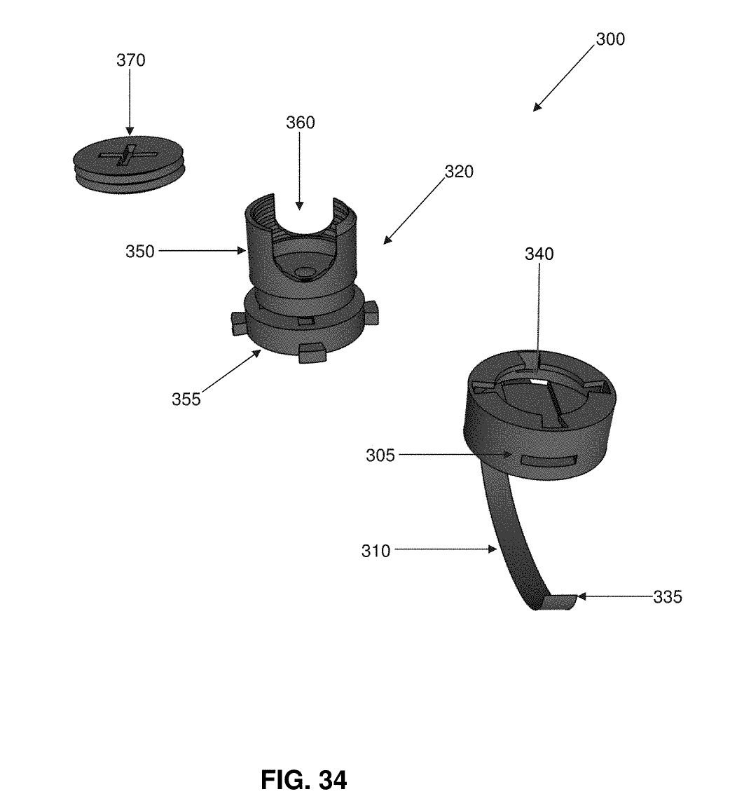

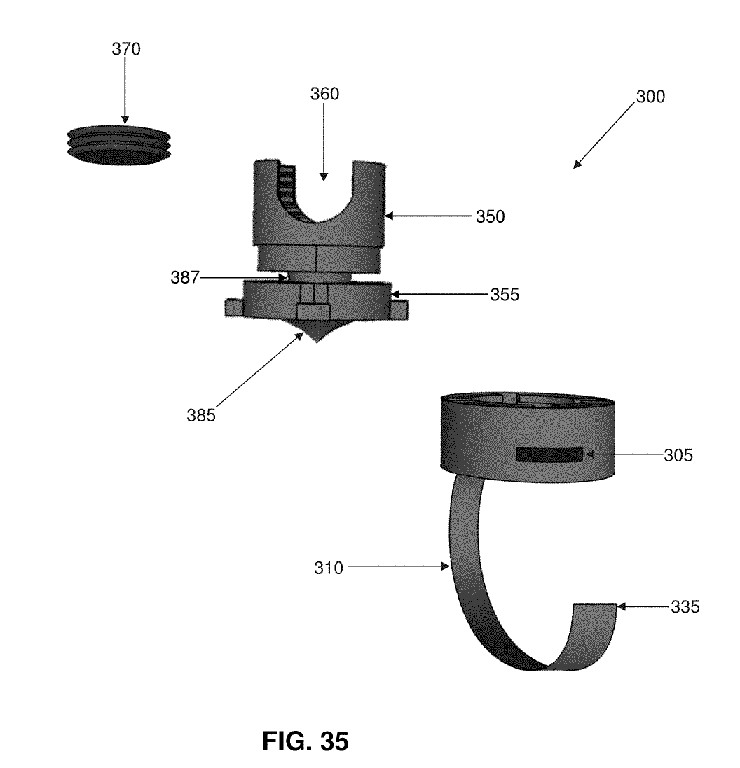

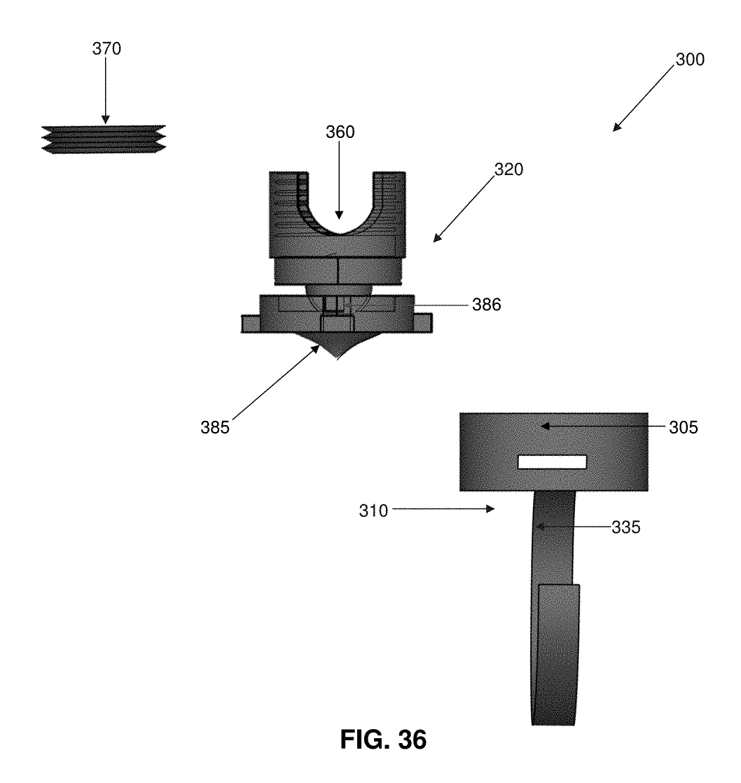

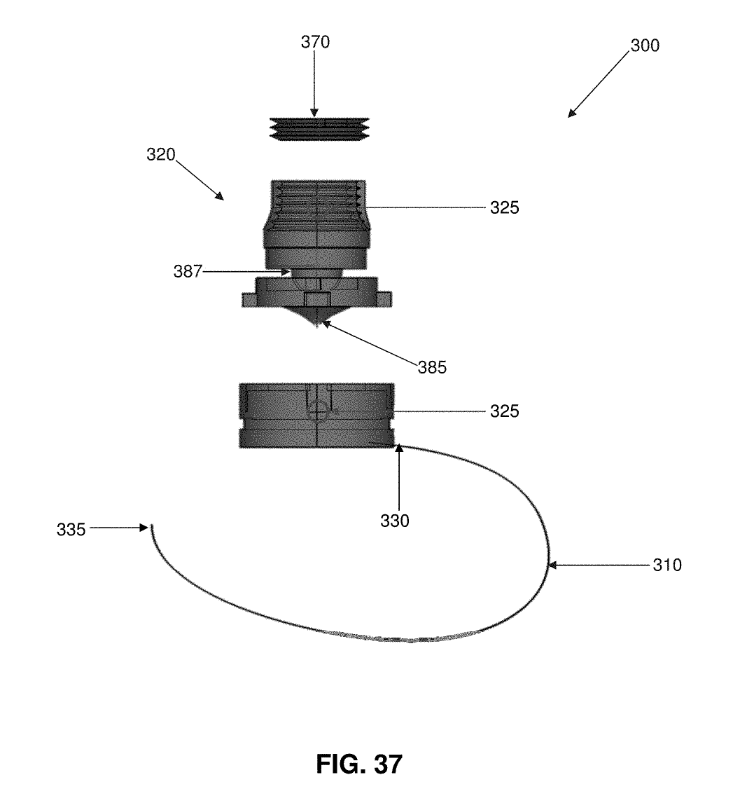

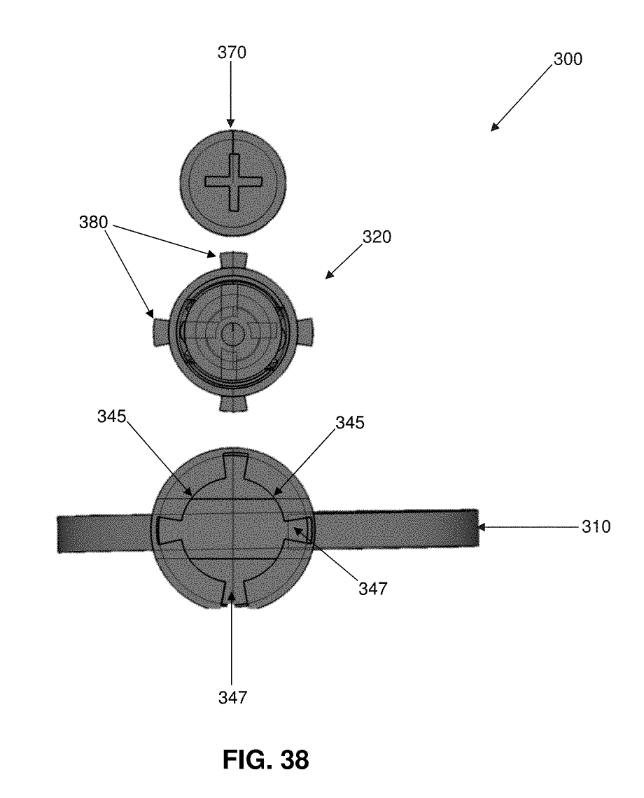

Looking next at FIGS. 33-38, there is shown another construction wherein one end of the sublaminar band is permanently fixed to the mount and the other end of the sublaminar band is selectively fixable to the mount.

More particularly, in this form of the invention, there is provided a mount 300 which generally comprises a bottom slot 305 for receiving the “free end” of a sublaminar band 310 and a bayonet mount 315 disposed adjacent to bottom slot 305 for receiving a fastener 320 (for securing sublaminar band 310 to mount 300).

Mount 300 also comprises one or more connection elements 325 (e.g., a recess, a flange, etc.) for connection to a band tensioning tool (see above) and/or a rod reduction tool (see above). In one preferred form of the invention, connection elements 325 comprise industry-standard connection elements of the sort used to connect a band tensioning tool and/or a rod reduction tool to a conventional mount (or head) of a pedicle-based spinal fixation system.

Sublaminar band 310 comprises a fixed end 330 and a “free end” 335. Fixed end 330 of sublaminar band 310 is permanently fixed to mount 300. Free end 335 of sublaminar band 310 is configured to be received by bottom slot 305 of mount 300 and selectively held in bottom slot 305 by fastener 320. In one preferred form of the present invention, sublaminar band 310 comprises a strong, flexible biocompatible material. By way of example but not limitation, sublaminar band 310 may comprise a polyester band, a Kevlar band, a braided titanium cable, etc.

In one preferred form of the invention, free end 335 of sublaminar band 310 may be stiffened somewhat so as to facilitate passage of free end 335 (and thus sublaminar band 310) around the lamina and into bottom slot 305 of mount 300. At the same time, free end 335 of sublaminar band 310 remains sufficiently flexible so that it can still be configured into a C-shaped loop. It should be appreciated that free end 335 of sublaminar band 310 may be stiffened in a variety of ways, e.g., by placing a stiffening material inside free end 335, by placing a stiffening material over free end 335, etc.

Bayonet mount 315 is configured to receive fastener 320 so that fastener 320 can secure free end 335 of sublaminar band 310 in mount 300. More particularly, bayonet mount 315 comprises a recess 340, a plurality of flanges 345, and a plurality of openings 347 disposed between the plurality of flanges 345.

Significantly, in this form of the invention, fastener 320 comprises a top portion 350 and a bottom portion 355. Top portion 350 generally comprises a U-shaped slot 360 for receiving spinal fixation rod 20 (FIG. 4), a threaded bore 365 disposed about U-shaped slot 360 for receiving a headless screw 370 (for securing spinal fixation rod 20 to fastener 320), and a hole 375. Bottom portion 355 generally comprises a plurality of tabs 380, a spike 385, and a non-circular recess 386. In one form of the invention, spike 385 may be replaced by a blunt element. If desired, the blunt element may comprise a projection, teeth, ridges, an abrasive region, etc. in order to increase the securement of the blunt element to sublaminar band 310 in bottom slot 305.

The plurality of tabs 380 are sized so as to be passable through the plurality of openings 347 of bayonet mount 315 and rotatable into recess 340 of bayonet mount 315 so that the plurality of tabs 380 may be locked below the plurality of flanges 345 of bayonet mount 315. Top portion 350 and bottom portion 355 are secured to one another via a rotatable connection 387 so as to allow top portion 350 and bottom portion 355 to rotate independently of one another. It will be appreciated that top portion 350 and bottom portion 355 become locked in position once spinal fixation rod 20 is secured to mount 300. Non-circular recess 386 in bottom portion 355 (accessible via hole 375 in top portion 350) may be used to turn bottom portion 355 without turning top portion 350 (see below).

In another form of the invention, top portion 350 and bottom portion 355 are locked to one another at the time of manufacture (i.e., top portion 350 and bottom portion 355 are not able to rotate independently of one another). In this form of the invention, additional tabs 280 in bottom portion 355, and additional openings 347 in bayonet mount 315, may be provided so as to facilitate proper alignment of U-shaped slot 360 and bottom slot 305 during spinal rod fixation.

Headless screw 370 is sized so as to fit into threaded bore 365 of U-shaped slot 360. A non-circular recess 388 (e.g., a cruciform recess) may be used to turn headless screw 370 (see below).

In use, mount 300 is first positioned adjacent to the lamina of a vertebra (or other anatomical or prosthetic structure) and free end 335 of sublaminar band 310 is passed around the lamina of the vertebra (or other anatomical or prosthetic structure). Then free end 335 of sublaminar band 310 is passed through bottom slot 305 of mount 300.

Once sublaminar band 310 has been passed around the lamina of a vertebra (or other anatomical or prosthetic structure) and free end 335 of sublaminar band 310 has been passed through bottom slot 305 of mount 300, sublaminar band 310 is tensioned and secured to mount 300. More particularly, an appropriate band tensioning tool 60 (see above) is used to tension free end 335 of sublaminar band 310. Then bottom portion 355 of fastener 320 is inserted into bayonet mount 315, with the plurality of tabs 380 of bottom portion 355 of fastener 320 aligned with the plurality of openings 347 of bayonet mount 315. The plurality of tabs 380 of bottom portion 355 of fastener 320 are then inserted through the plurality of openings 347 of bayonet mount 315 so that the plurality of tabs 380 of bottom portion 355 of fastener 320 lie in recess 340 of bayonet mount 315. Once the plurality of tabs 380 of bottom portion 355 of fastener 320 are in recess 340 of bayonet mount 315, bottom portion 355 of fastener 320 is turned (e.g., by accessing non-circular recess 386 in bottom portion 355 via hole 375 of top portion 350) so as to position the plurality of tabs 380 of bottom portion 355 of fastener 320 beneath the plurality of flanges 345 of bayonet mount 315, thereby locking fastener 320 to bayonet mount 315, and thus locking fastener 320 to mount 300. When bayonet mount 315 and fastener 320 are locked in this position, spike 385 of bottom portion 355 of fastener 320 presses into sublaminar band 310 so as to secure the tensioned sublaminar band 310 to mount 300. It should be appreciated that where spike 385 is replaced with a blunt element, the blunt element compresses sublaminar band 310 in bottom slot 305 so as to secure the tensioned sublaminar band 310 to mount 300.

Note that sublaminar band 310 is tensioned to the lamina (or other anatomical or prosthetic structure) and mount 300, and then secured to the lamina and mount 300, independently of any reduction of spinal fixation rod 20 to mount 300 (or any reduction of mount 300 to spinal rod 20).

If desired, sublaminar band 310 may then be trimmed to length by removing any excess of free end 335 extending from bottom slot 305 of mount 300.

Next, spinal fixation rod 20 is reduced and secured to mount 300. This is done by reducing spinal fixation rod 20 into U-shaped slot 360 of top portion 350 of fastener 320. Note that top portion 350 can be rotated relative to bottom portion 355 so as to ensure that U-shaped slot 360 is properly oriented relative to spinal fixation rod 20. Once spinal fixation rod 20 has been reduced into U-shaped slot 360 of top portion 350 of fastener 320, headless screw 370 may be rotatably advanced (e.g., using non-circular recess 387) into threaded bore 365 of U-shaped slot 360 of fastener 320 so as to secure spinal fixation rod 20 to screw mount 320, and thus secure spinal fixation rod 20 to mount 300. Note that spinal fixation rod 20 is reduced to mount 300 (or mount 300 reduced to spinal fixation rod 20), and then the spinal fixation rod is secured to mount 300, independently of any tensioning and securing of sublaminar band 310 to mount 300. Indeed, sublaminar band 310 does not need to be engaged in any way by the surgeon and/or his/her instruments during the time that the spinal fixation rod 20 is being reduced to mount 300 (or vice versa).

If sublaminar band 310 has not already been trimmed to length, it may be trimmed to length at this point in the procedure.

It should be appreciated that since the mounts 300 can be independently and initially tensioned and locked to the spine, the surgeon can obtain all fixation points to the spine in a finalized stable configuration (for example, pedicle screws, hooks and sublaminar implants) prior to the insertion and/or reduction of the spinal fixation rod to the fixation points.

It should also be appreciated that inasmuch as mount 300 has the connection mechanism for spinal fixation rod 20 (i.e., U-shaped slot 360 and threaded bore 365/headless screw 370) disposed “in-line” (i.e., in vertical alignment) with the connection mechanism for sublaminar band 310 (i.e., bottom slot 305 and bayonet mount 315/fastener 320), mount 300 can have a smaller “footprint” which allows the surgeon greater latitude in proper anatomical positioning of the mounts 300.

It should also be appreciated that, if desired, the connection mechanism for spinal fixation rod 20 (i.e., U-shaped slot 360 and threaded bore 365/headless screw 370) need not be aligned with the connection mechanism for sublaminar band 310 (i.e., bottom slot 305 and bayonet mount 315/fastener 320). If desired, U-shaped slot 360 may be laterally offset from the axis of bayonet mount 315 in order to accommodate anatomical needs.

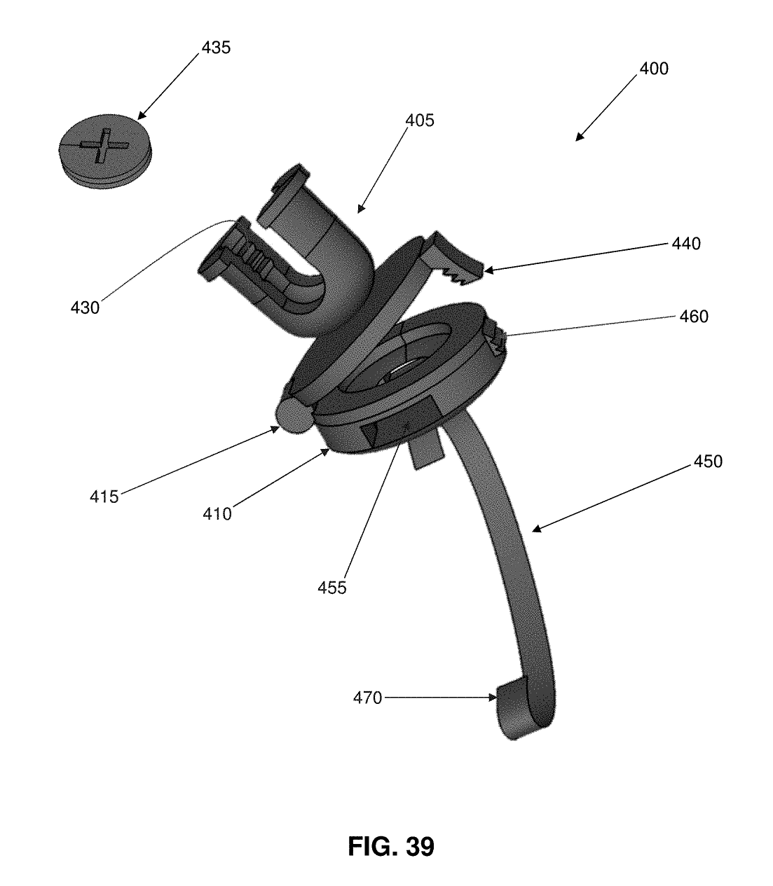

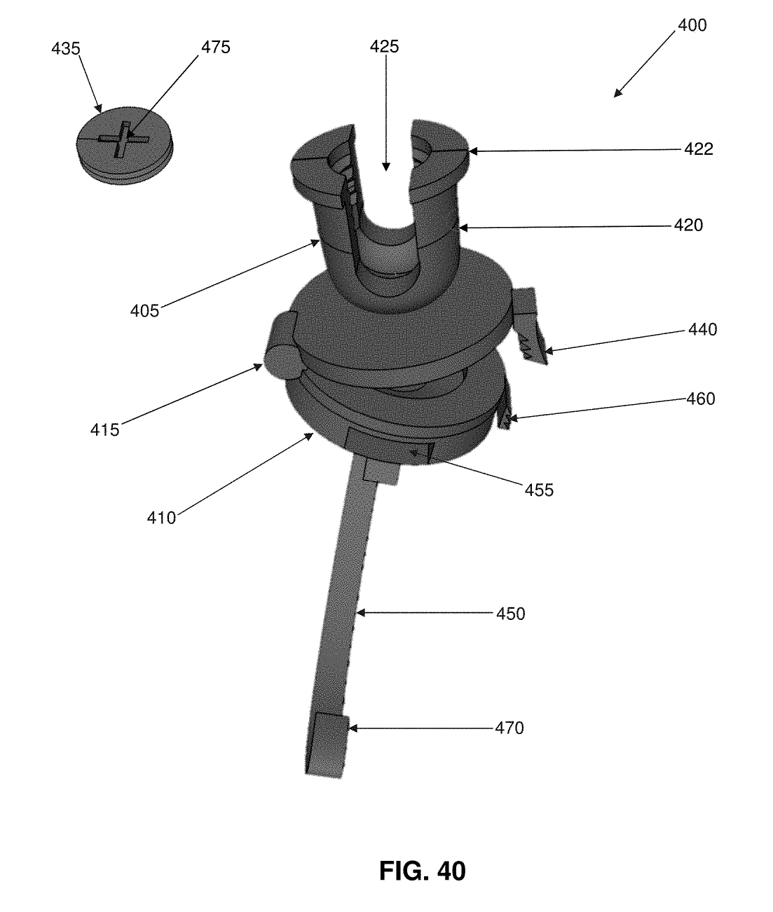

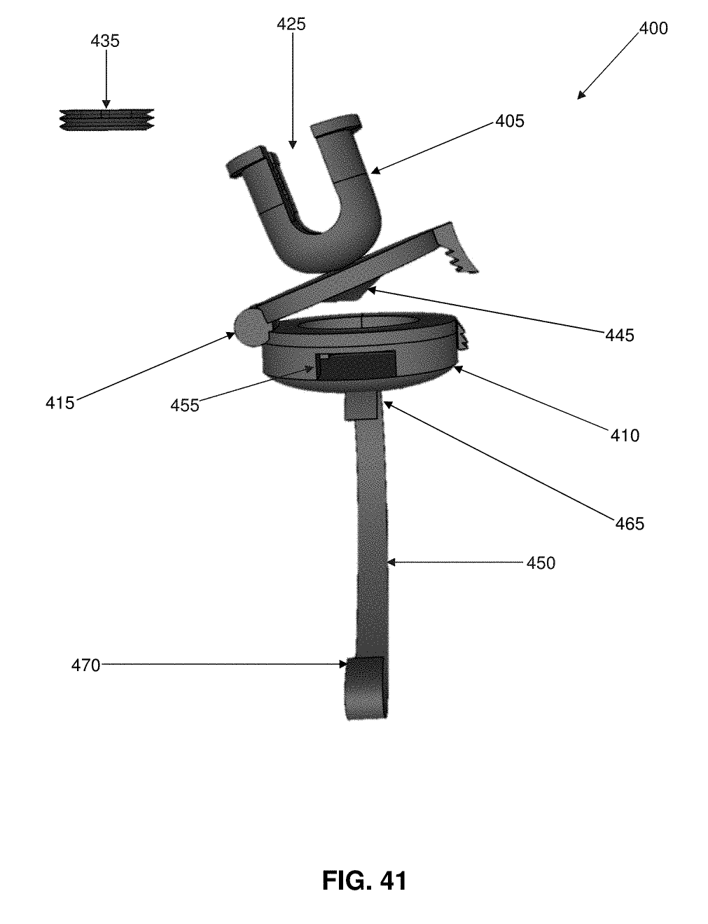

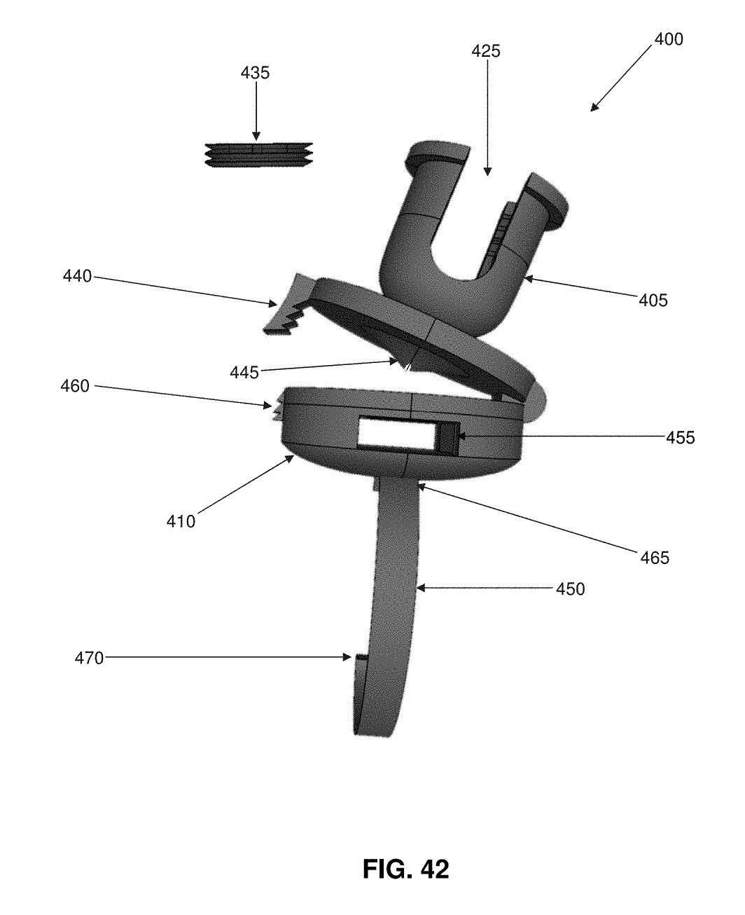

Looking next at FIGS. 39-42, there is shown another construction wherein one end of the sublaminar band is permanently fixed to the mount and the other end of the sublaminar band is selectively fixable to the mount.

More particularly, in this form of the invention, there is provided a mount 400 which generally comprises a top portion 405, a bottom portion 410, and a pivot mount 415 pivotally connecting top portion 405 and bottom portion 410.

Mount 400 also comprises one or more connection elements 420 (e.g., a recess, a flange, etc.) for connection to a band tensioning tool (see above) and/or a rod reduction tool (see above). In one preferred form of the invention, connection elements 420 comprise industry-standard connection elements of the sort used to connect a band tensioning tool and/or a rod reduction tool to a conventional mount (or head) of a pedicle-based spinal fixation system. In another preferred form of the invention, connection element 420 comprises a lip 422 (or other connection structure) on top portion 405 of mount 400 for connection with a band tensioning tool and/or a rod reduction tool.

Top portion 405 generally comprises a U-shaped slot 425 for receiving spinal fixation rod 20 (FIG. 4), a threaded bore 430 disposed adjacent to U-shaped slot 425 for receiving a headless screw 435, a clasp 440 and a spike 445. In one form of the invention, spike 445 may be replaced by a blunt element. If desired, the blunt element may comprise a projection, teeth, ridges, an abrasive region, etc. in order to increase the securement of the blunt element to sublaminar band 450.

Bottom portion 410 has one end of sublaminar band 450 permanently fixed thereto and receives the other end of the sublaminar band for selectively fixating the same. More particularly, bottom portion 410 generally comprises a bottom slot 455 for receiving the “free end” of sublaminar band 450. Bottom portion 410 also comprises ridges 460 for connection with clasp 440 of top portion 405.

Pivot mount 415 pivotally connects top portion 405 to bottom portion 410 and allows top portion 405 to move relative to bottom portion 410 (i.e., to open and close relative to bottom portion 410).

Sublaminar band 450 comprises a fixed end 465 and a free end 470. Fixed end 465 of sublaminar band 450 is secured to bottom portion 410 of mount 400. Free end 470 of sublaminar band 450 is configured to be received by bottom slot 455 of bottom portion 410 of mount 400. In one preferred form of the present invention, sublaminar band 450 comprises a strong, flexible biocompatible material. By way of example but not limitation, sublaminar band 450 may comprise a polyester band, a Kevlar band, a braided titanium cable, etc.

In one preferred form of the invention, free end 470 of sublaminar band 450 may be stiffened somewhat so as to facilitate passage of free end 470 (and thus sublaminar band 450) around the lamina and into bottom slot 455 of mount 400. At the same time, free end 470 of sublaminar band 450 remains sufficiently flexible so that it can still be configured into a C-shaped loop. It should be appreciated that free end 470 of sublaminar band 450 may be stiffened in a variety of ways, e.g., by placing a stiffening material inside free end 470, by placing a stiffening material over free end 470, etc.

It should be appreciated that when mount 400 is in its “closed” position (i.e., clasp 440 of top portion 405 is clasped onto ridges 460 of bottom portion 410), spike 445 of top portion 405 penetrates (or, if spike 445 is replaced by a blunt element, compresses) the free end 470 of a sublaminar band 450 disposed in bottom slot 455 of bottom portion 410 so as to secure the free end 470 of sublaminar band 450 relative to mount 400. Conversely, when mount 400 is in its “open” position (i.e., when top portion 405 is spaced away from bottom portion 410), spike 445 of top portion 405 does not penetrate (or, if spike 445 is replaced by a blunt element, compresses) the free end 470 of a sublaminar band 450 disposed in bottom slot 455 of bottom portion 410 and the free end 470 of sublaminar band 450 is free to move in bottom slot 455 of bottom portion 410.

Headless screw 435 is sized so as to fit into threaded bore 430. A non-circular recess 475 (e.g., a cruciform recess) may be used to turn headless screw 435 (see below).

In use, mount 400 is first positioned adjacent to the lamina of a vertebra (or other anatomical or prosthetic structure), top portion 405 is opened relative to bottom portion 410, free end 470 of sublaminar band 450 is passed around the lamina of the vertebra (or other anatomical or prosthetic structure), and then free end 470 of sublaminar band 450 is passed through bottom slot 455 of bottom portion 410 of mount 400.

Once sublaminar band 450 has been passed around the lamina of the vertebra (or other anatomical or prosthetic structure) and free end 470 of sublaminar band 450 has been passed through bottom slot 455 of mount 400, sublaminar band 450 is tensioned and secured to mount 400. More particularly, an appropriate band tensioning tool 60 (see above) is used to tension the sublaminar band. Then mount 400 is “closed”, i.e., top portion 405 is pivoted into alignment with bottom portion 410 so that clasp 440 of top portion 405 clasps onto ridges 460 of bottom portion 410. It should be appreciated that when mount 400 is closed, spike 445 of top portion 405 presses into sublaminar band 450 so as to secure the tensioned sublaminar band 450 to mount 400. It should be appreciated that where spike 445 is replaced with a blunt element, the blunt element compresses sublaminar band 450 in bottom slot 455 so as to secure the tensioned sublaminar band 450 to mount 400.

Note that sublaminar band 450 is tensioned to the lamina (or other anatomical or prosthetic structure) and mount 400, and then secured to the lamina and mount 400, independently of any reduction of spinal fixation rod 20 to mount 400 (or any reduction of mount 400 to spinal rod 20).

If desired, sublaminar band 450 may then be trimmed to length by removing any excess of free end 470 extending from bottom slot 455 of mount 400.

Next, spinal fixation rod 20 is reduced and secured to mount 400. This is done by reducing spinal fixation rod 20 into U-shaped slot 425 of top portion 405 of mount 400. Once spinal fixation rod 20 has been reduced into U-shaped slot 425 of top portion 405 of mount 400, headless screw 435 may be advanced into threaded bore 430 of U-shaped slot 425 so as to secure spinal fixation rod 20 to mount 400. Note that spinal fixation rod 20 is reduced to mount 400 (or mount 400 reduced to spinal fixation rod 20), and then the spinal fixation rod is secured to mount 400, independently of any tensioning and securing of sublaminar band 450 to mount 400. Indeed, sublaminar band 450 does not need to be engaged in any way by the surgeon and/or his/her instruments during the time that the spinal fixation rod 20 is being reduced to mount 400 (or vice versa).

If sublaminar band 450 has not already been trimmed to length, it may be trimmed to length at this point in the procedure.

It should be appreciated that since the mounts 400 can be independently and initially tensioned and locked to the spine, the surgeon can obtain all fixation points to the spine in a finalized stable configuration (for example, pedicle screws, hooks and sublaminar implants) prior to the insertion and/or reduction of the spinal fixation rod to the fixation points.

It should also be appreciated that inasmuch as mount 400 has the connection mechanism for spinal fixation rod 20 (i.e., U-shaped slot 425 and threaded bore 430/headless screw 435) disposed “in-line” (i.e., in vertical alignment) with the connection mechanism for sublaminar band 450 (i.e., bottom slot 455 and spike 445), mount 400 can have a smaller “footprint” which allows the surgeon greater latitude in proper anatomical positioning of the mounts 400.

It should also be appreciated that, if desired, the connection mechanism for spinal fixation rod 20 (i.e., U-shaped slot 425 and threaded bore 430/headless screw 435) need not be aligned with the connection mechanism for sublaminar band 450 (i.e., bottom slot 455 and spike 445). If desired, U-shaped slot 425 may be laterally offset from the axis of bottom slot 455 in order to accommodate anatomical needs.

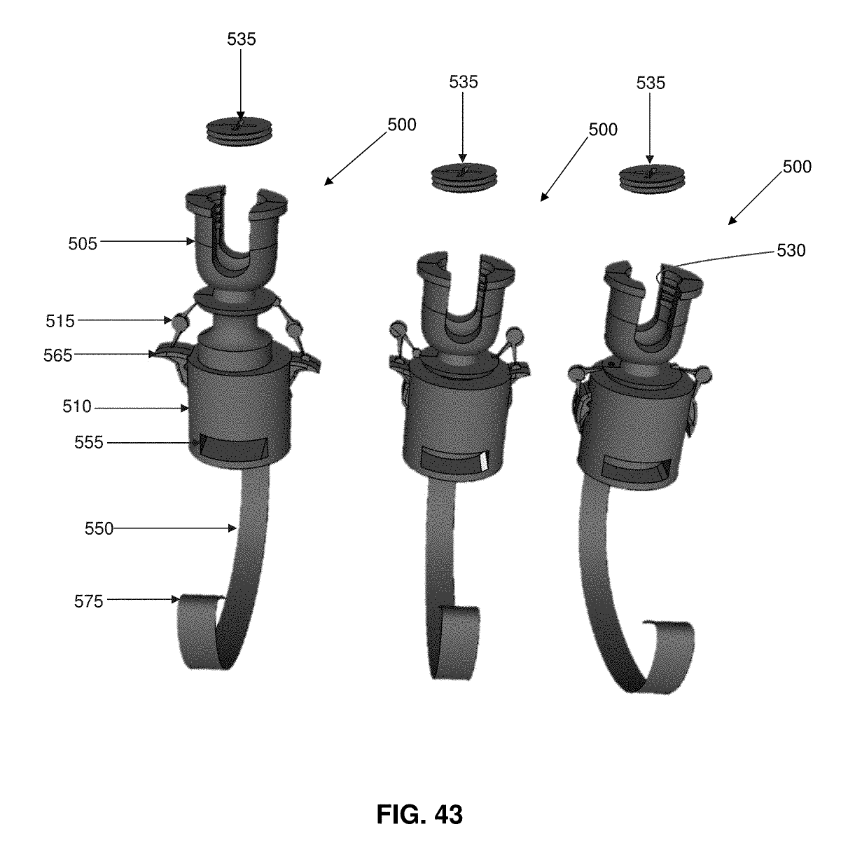

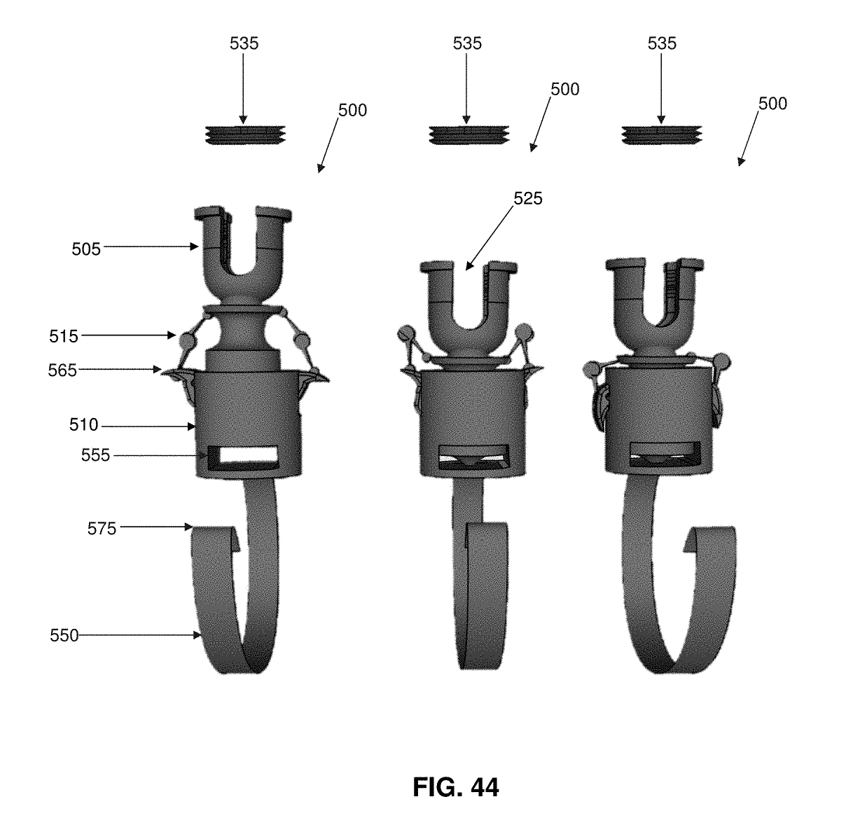

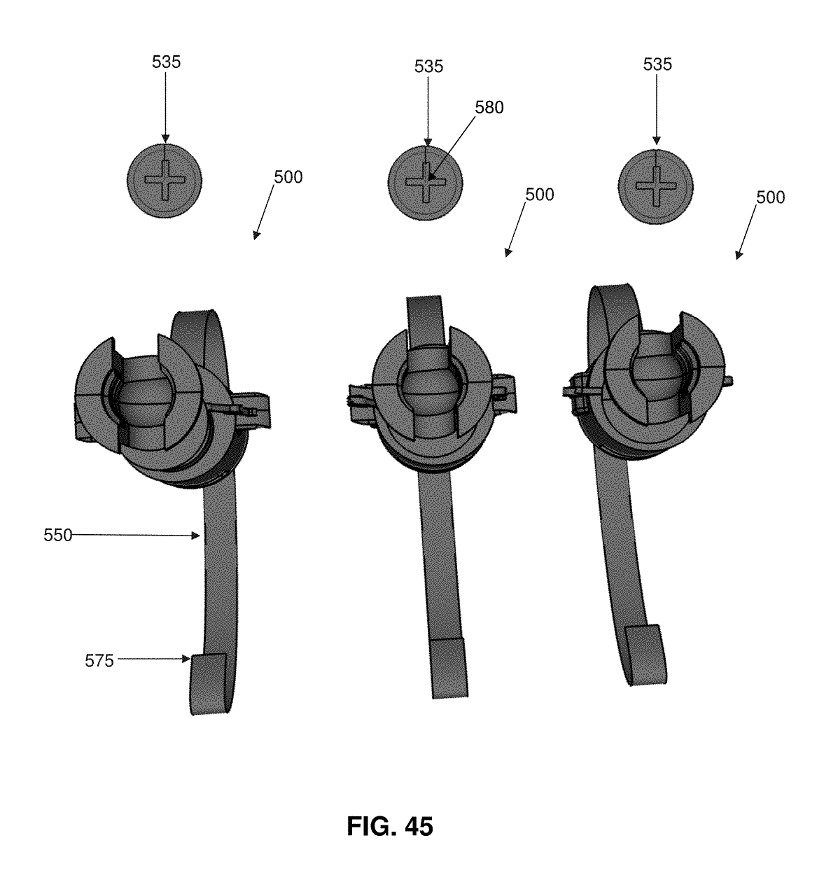

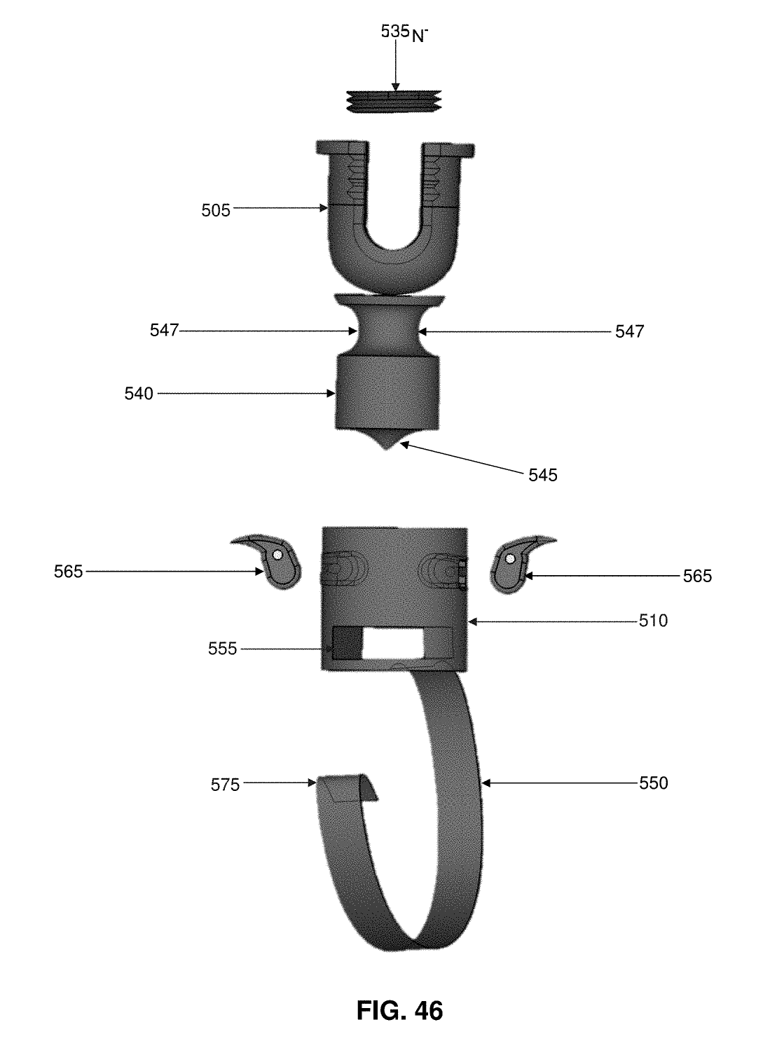

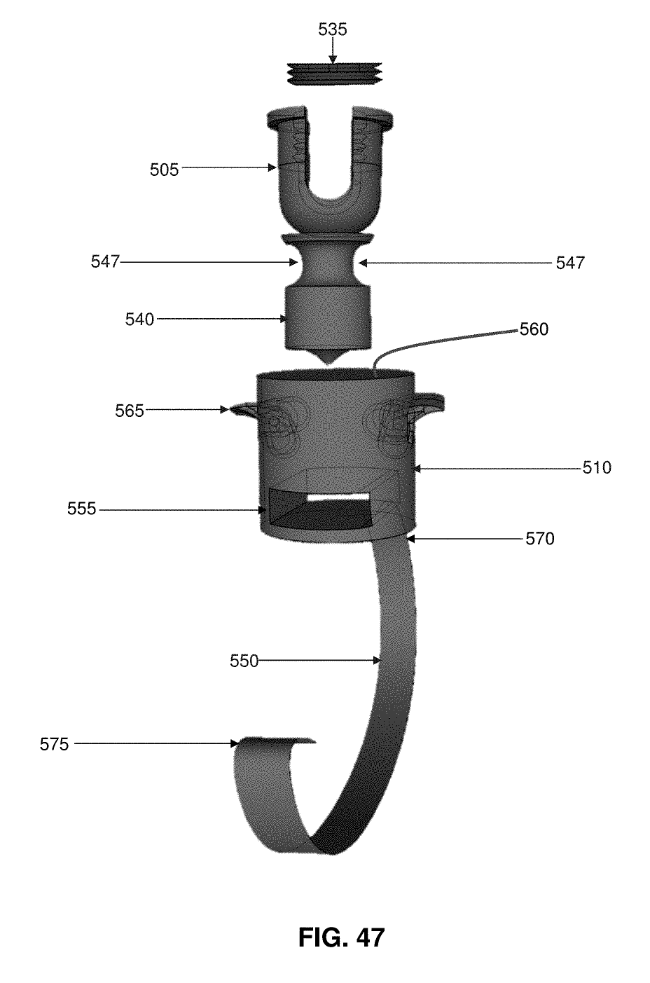

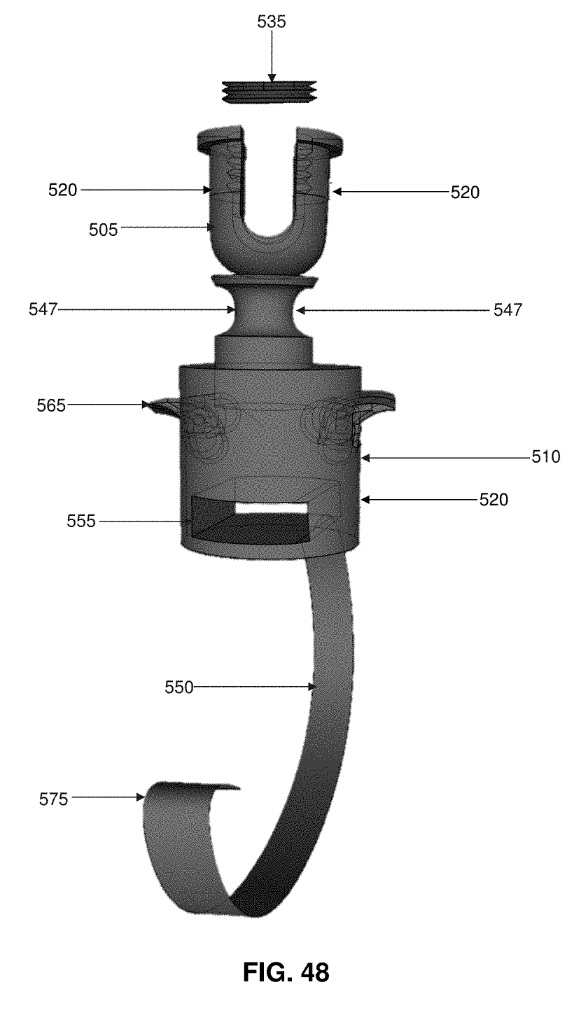

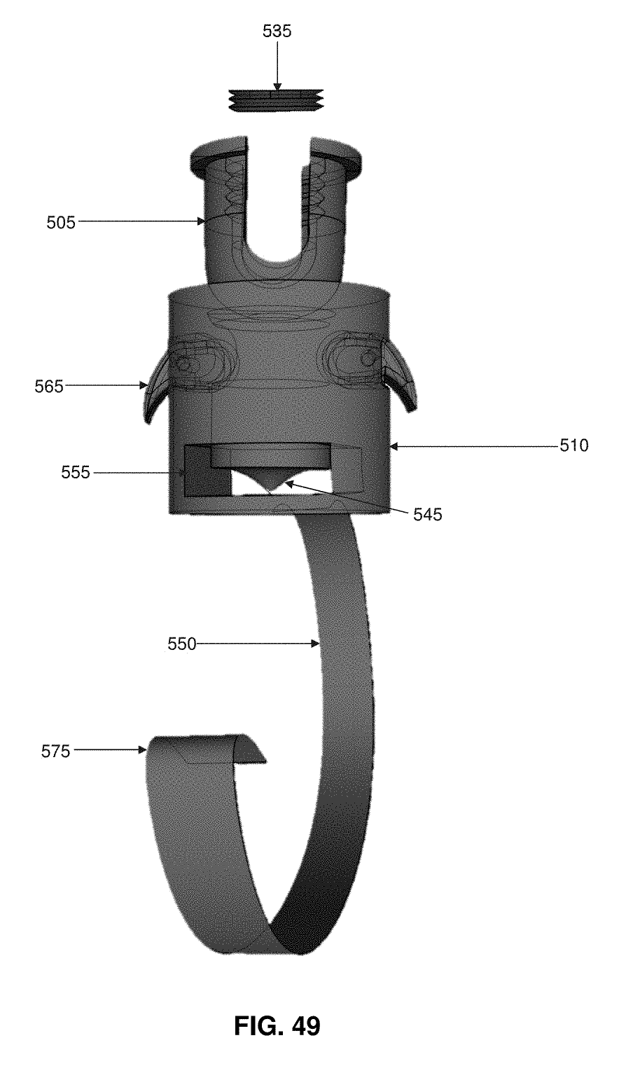

Looking next at FIGS. 43-49, there is shown another construction wherein one end of the sublaminar band is permanently fixed to the mount and the other end of the sublaminar band is selectively fixable to the mount.

More particularly, in this form of the invention, there is provided a mount 500 which generally comprises a top portion 505, a bottom portion 510, and a pair of hinges 515 connecting top portion 505 to bottom portion 510.

Mount 500 also comprises one or more connection elements 520 (e.g., a recess, a flange, etc.) for connection to a band tensioning tool (see above) and/or a rod reduction tool (see above). In one preferred form of the invention, connection elements 520 comprise industry-standard connection elements of the sort used to connect a band tensioning tool and/or a rod reduction tool to a conventional mount (or head) of a pedicle-based spinal fixation system.

Top portion 505 generally comprises a U-shaped slot 525 for receiving spinal fixation rod 20 (FIG. 4), a threaded bore 530 disposed adjacent to U-shaped slot 525 for receiving a headless screw 535, a body 540, and a spike 545 extending from the bottom end of body 540. Body 540 comprises a pair of recesses 547. In one form of the invention, spike 545 may be replaced by a blunt element. If desired, the blunt element may comprise a projection, teeth, ridges, an abrasive region, etc. in order to increase the securement of the blunt element to sublaminar band 550.

Bottom portion 510 has one end of sublaminar band 550 permanently fixed thereto and receives the other end of the sublaminar band for selectively fixating the same. More particularly, bottom portion 510 generally comprises a bottom slot 555, a recess 560, and a pair of arms 565 connected to hinges 515.

Hinges 515 are connected to arms 565 of bottom portion 510. Hinges 515 move in conjunction with top portion 505. More particularly, when top portion 505 is pushed downward, hinges 515 flex. When top portion 505 is pushed downward into bottom portion 510, arms 565 may be manually moved inboard so as to advance arms 565 into recesses 547 of body 540, whereby to engage body 540 and lock body 540 relative to bottom portion 510. Conversely, arms 565 may be manually moved outboard so as to remove arms 565 from recesses 547 of body 540, whereby to release body 540 relative to bottom portion 510. Top portion 505 may then be pulled upward, with hinges 515 flexing.

Sublaminar band 550 comprises a fixed end 570 and a free end 575. Fixed end 570 of sublaminar band 550 is secured to bottom portion 510 of mount 500. Free end 575 of sublaminar band 550 is configured to be received by bottom slot 555 of bottom portion 510 of mount 500. In one preferred form of the present invention, sublaminar band 550 comprises a strong, flexible biocompatible material. By way of example but not limitation, sublaminar band 550 may comprise a polyester band, a Kevlar band, a braided titanium cable, etc.

In one preferred form of the invention, free end 575 of sublaminar band 550 may be stiffened somewhat so as to facilitate passage of free end 575 (and thus sublaminar band 550) around the lamina and into bottom slot 555 of mount 500. At the same time, free end 575 of sublaminar band 550 remains flexible so that it can still be configured into a C-shaped loop. It should be appreciated that free end 575 of sublaminar band 550 may be stiffened in a variety of ways, e.g., by placing a stiffening material inside free end 575, by placing a stiffening material over free end 575, etc.

It should be appreciated that when mount 500 is in its “open” position (i.e., arms 565 are positioned in their outboard position, free from recesses 547 of body 540), free end 570 of sublaminar band 550 is free to pass through bottom slot 555 of bottom portion 510. Conversely, when mount 500 is in its “closed” position (i.e., arms 565 are positioned in their inboard position, locked in recesses 547 of body 540), spike 545 of top portion 505 projects into (or, if spike 545 is a blunt element, compresses) free end 575 of sublaminar band 550 in bottom slot 555 of bottom portion 510 and locks sublaminar band 550 relative to mount 500.

Headless screw 535 is sized so as to fit into threaded bore 530. A non-circular recess 580 (e.g., a cruciform recess) may be used to turn headless screw 535 (see below).

In use, mount 500 is first positioned adjacent to the lamina of a vertebra (or other anatomical or prosthetic structure), top portion 505 is opened relative to bottom portion 510, free end 575 of sublaminar band 550 is passed around the lamina of the vertebra (or other anatomical or prosthetic structure), and free end 575 of sublaminar band 550 is passed through bottom slot 555 of bottom portion 510 of mount 500.

Once sublaminar band 550 has been passed around the lamina of the vertebra (or other anatomical or prosthetic structure) and free end 575 of sublaminar band 550 has been passed through bottom slot 555 of mount 500, sublaminar band 550 is tensioned and secured to mount 500. More particularly, an appropriate band tensioning tool 60 (see above) is used to tension the sublaminar band. Then mount 500 is “locked” (i.e., top portion 505 is pushed downward, moving body 540 into recess 560, collapsing hinges 515, and then arms 565 of bottom portion 510 are manually moved inboard into their downward “locked” position in recesses 547 of body 540). When mount 500 is in its “locked” position, spike 545 of top portion 505 presses into sublaminar band 550 so as to secure the tensioned sublaminar band 550 to mount 500. It should be appreciated that where spike 545 is replaced with a blunt element, the blunt element compresses sublaminar band 550 in bottom slot 555 so as to secure the tensioned sublaminar band 550 to mount 500.

Note that sublaminar band 550 is tensioned to the lamina (or other anatomical or prosthetic structure) and mount 500, and then secured to the lamina and mount 500, independently of any reduction of spinal fixation rod 20 to mount 500 (or any reduction of mount 500 to spinal rod 20).

If desired, sublaminar band 550 may then be trimmed to length by removing any excess of free end 575 extending from mount 500.

Next, spinal fixation rod 20 is reduced and secured to mount 500. This is done by reducing spinal fixation rod 20 into U-shaped slot 525 of top portion 505 of mount 500. Once spinal fixation rod 20 has been reduced into U-shaped slot 525 of top portion 505 of mount 500, headless screw 535 may be advanced into threaded bore 530 of mount 500 so as to secure spinal fixation rod 20 to mount 500. Note that spinal fixation rod 20 is reduced to mount 500 (or mount 500 reduced to spinal fixation rod 20), and then the spinal fixation rod is secured to mount 500, independently of any tensioning and securing of sublaminar band 545 to mount 500. Indeed, sublaminar band 550 does not need to be engaged in any way by the surgeon and/or his/her instruments during the time that the spinal fixation rod 20 is being reduced to mount 500 (or vice versa).

If sublaminar band 550 has not already been trimmed to length, it may be trimmed to length at this point in the procedure.

It should be appreciated that since the mounts 500 can be independently and initially tensioned and locked to the spine, the surgeon can obtain all fixation points to the spine in a finalized stable configuration (for example, pedicle screws, hooks and sublaminar implants) prior to the insertion and/or reduction of the spinal fixation rod to the fixation points.

It should also be appreciated that inasmuch as mount 500 has the connection mechanism for spinal fixation rod 20 (i.e., U-shaped slot 525 and threaded bore 530/headless screw 535) disposed “in-line” (i.e., in vertical alignment) with the connection mechanism for sublaminar band 550 (i.e., bottom slot 555 and spike 545), mount 500 can have a smaller “footprint” which allows the surgeon greater latitude in proper anatomical positioning of the mounts 500.

It should also be appreciated that, if desired, the connection mechanism for spinal fixation rod 20 (i.e., U-shaped slot 525 and threaded bore 530/headless screw 535) need not be aligned with the connection mechanism for sublaminar band 550 (i.e., bottom slot 555 and spike 545). If desired, U-shaped slot 525 may be laterally offset from the axis of bottom slot 555 in order to accommodate anatomical needs.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for spinal repair, the method comprising:
positioning a band having a first end and a second end around at least a portion of a bone;
positioning a mount having independent first and second coupling members, the first coupling member for coupling the mount to the band and the second coupling member for coupling a spinal fixation rod to the mount;
coupling the band to the mount using the first coupling member;
tensioning at least one of the ends of the band;
while maintaining tension on the band, fixing the mount to the band using the first coupling member, wherein the first coupling member fixes the mount to the band by advancing toward the at least a portion of the bone; and
reducing the distance between the spinal fixation rod and the mount;
wherein the step of reducing the distance between the spinal fixation rod and the mount is achieved without tensioning the band; and
wherein the step of reducing the distance between the spinal fixation rod and the mount does not change the location or orientation of the mount relative to the bone.

2. A method according to claim 1 further comprising the step of fixing the spinal fixation rod to the mount using the second coupling member.

3. A method according to claim 1 further comprising correcting a pathology of the spine.

4. A method according to claim 1 further comprising measuring the tension of the band.

5. A method according to claim 1 wherein the band is only tensioned once and is not re-tensioned during the step of reducing the distance between the spinal fixation rod and the mount.

6. A method according to claim 1 further comprising positioning and tensioning more than one band coupled to a mount at more than one spine level.

7. A method according to claim 1 wherein the band and the mount are positioned and fixed in their final location prior to the reduction, insertion or coupling of the spinal fixation rod.

8. A method according to claim 1 wherein the distance between the rod and the mount is reduced without the band being engaged by the surgeon and/or his/her instruments.

9. A method according to claim 1 further comprising coupling one or more bands to the mount and positioning the one or more bands around different bone structures such that the mounts are fixed into their final stable position to the spine prior to rod reduction, insertion or coupling to the mounts.

10. A method according to claim 1 wherein the second coupling member is laterally offset from the first coupling member.

11. A spinal fixation system, the spinal fixation system comprising:
a band;
a spinal fixation rod; and
a mount, wherein the mount comprises a first connection mechanism for connecting the band to the mount, and a second connection mechanism for connecting the spinal fixation rod to the mount, wherein the first and second connection mechanisms operate independently of one another, and further wherein the first connection mechanism connects the band to the mount by advancing a member away from the second connection mechanism, and further wherein the first connection mechanism and the second connection mechanism are disposed in vertical alignment with one another.

12. A spinal fixation system, the spinal fixation system comprising:
a mount;
a band having first and second ends, wherein the first end of the band is fixed to the mount and the second end of the band comprises a free end selectively fixable to the mount; and
a spinal fixation rod;
wherein the mount comprises a first connection mechanism for connecting the second end of the band to the mount; and
wherein the mount comprises a second connection mechanism for connecting the spinal fixation rod to the mount.

13. A spinal fixation system according to claim 12 wherein the mount comprises a cavity for receiving the first connection mechanism, and further wherein the first connection mechanism is configured to be selectively rotated within the cavity so as to secure the first connection mechanism to the mount, whereby to secure the second end of the band to the mount.

14. A spinal fixation system according to claim 13 wherein the first connection mechanism comprises a plurality of projections, and wherein the mount comprises a plurality of flanges disposed about the cavity, such that when the first connection mechanism is disposed within the cavity of the mount, the plurality of projections on the first connection mechanism are engaged by the plurality of flanges, whereby to secure the first connection mechanism to the mount.

15. A spinal fixation system according to claim 13 wherein the first connection mechanism comprises an engaging element directed away from the first connection mechanism toward the second end of the band, such that when the first connection mechanism is secured to the mount, the engaging element engages the second end of the band, whereby to secure the second end of the band to the mount.

16. A spinal fixation system according to claim 12 wherein the mount comprises a cavity for receiving the first connection mechanism, wherein the first connection mechanism is configured to be selectively rotated within the cavity so as to secure the first connection mechanism to the mount, whereby to secure the second end of the band to the mount, and further wherein the second connection mechanism is mounted to the first connection mechanism.

17. A spinal fixation system according to claim 16 wherein the second connection mechanism is rotatably mounted to the first connection mechanism.

18. A spinal fixation system according to claim 12 wherein the first connection mechanism comprises a hinge for hingedly mounting the first connection mechanism to the mount, and further wherein the first connection mechanism is configured to be selectively rotated relative to the mount on the hinge such that the first connection mechanism is disengaged from the second end of the band when the hinge is in the open position, and such that the first connection mechanism engages the second end of the band when the hinge is in the closed position.

19. A spinal fixation system according to claim 18 wherein the first connection mechanism comprises a clasp, wherein the mount comprises ridges for engagement by the clasp, and further wherein when the hinge is in the closed position, the clasp engages the ridges, whereby to lock the first connection mechanism to the mount.

20. A spinal fixation system according to claim 18 wherein the first connection mechanism comprises an engaging element directed away from the first connection mechanism toward the second end of the band, such that when the hinge is in the closed position, the engaging element engages the second end of the band, whereby to secure the second end of the band to the mount.

21. A spinal fixation system according to claim 12 wherein the mount comprises a cavity for receiving at least a portion of the first connection mechanism, wherein the first connection mechanism is hingedly mounted to the mount by two opposing hinges, such that the first connection mechanism can be selectively advanced relative to the mount so as to dispose at least a portion of the first connection mechanism within the cavity, whereby to engage the second end of the band to the mount with the first connection mechanism.

22. A spinal fixation system according to claim 21 wherein the mount further comprises a pair of pivotally mounted arms for selectively engaging the first connection mechanism when at least a portion of the first connection mechanism is disposed in the cavity, and further wherein when the pair of pivotally mounted arms engage the first connection mechanism, the second end of the band is fixed to the mount.

23. A method for spinal repair, the method comprising:
providing a spinal fixation system, the spinal fixation system comprising:
a mount;
a band having first and second ends, wherein the first end of the band is fixed to the mount and the second end of the band comprises a free end selectively fixable to the mount; and
a spinal fixation rod;
wherein the mount comprises a first connection mechanism for connecting the second end of the band to the mount; and
wherein the mount comprises a second connection mechanism for connecting the spinal fixation rod to the mount;
passing the second end of the band around at least a portion of a bone;
connecting the second end of the band to the mount using the first connection mechanism so as to secure the mount to the bone; and
connecting the spinal fixation rod to the mount using the second connection mechanism.
